(19) United States
(12) Patent Application Publication
(10) Pub. No.: US 2026/0043000 A1
(43) Pub. Date: Feb. 12, 2026

(54) RECOMBINANT STRAIN FOR PRODUCING L-AMINO ACID, CONSTRUCTION METHOD THEREFOR, AND APPLICATION THEREOF

(71) Applicant: INNER MONGOLIA EPPEN BIOTECH CO., LTD., Inner Mongolia (CN)

(72) Inventors: Aiying WEI, Inner Mongolia (CN); Gang MENG, Inner Mongolia (CN); Xiaoqun ZHOU, Inner Mongolia (CN); Chunguang ZHAO, Inner Mongolia (CN); Fengyong MA, Inner Mongolia (CN); Huiping JIA, Inner Mongolia (CN); Lipeng YANG, Inner Mongolia (CN); Houbo SU, Inner Mongolia (CN); Xiaowei GUO, Inner Mongolia (CN); Bin TIAN, Inner Mongolia (CN); Xiaohang GAO, Inner Mongolia (CN)

(73) Assignee: INNER MONGOLIA EPPEN BIOTECH CO., LTD., Inner Mongolia (CN)

(21) Appl. No.: 19/366,448

(22) Filed: Oct. 22, 2025

Related U.S. Application Data
(62) Division of application No. 18/001,218.

(30) Foreign Application Priority Data
Jun. 8, 2020 (CN) 202010514037.1
Aug. 7, 2020 (CN) 202010790887.4
Oct. 15, 2020 (CN) 202011105063.5

(51) Int. Cl. C12N 1/20; C12N 15/77; C12P 13/04; C12R 1/15

(52) U.S. Cl. CPC C12N 1/205; C12N 15/77; C12P 13/04; C12R 2001/15

(57) ABSTRACT

A bacterium for producing L-amino acid has improved expression of a polynucleotide encoding a protein represented by SEQ ID NO:3 and improved expression of a polynucleotide encoding a protein represented by SEQ ID NO:31, and/or has mutations in bases at positions −45 bp and −47 bp of a promotor region represented by SEQ ID NO:57. A polynucleotide encodes proteins and can be included in a recombinant vector, which can be included in a recombinant strain. These are useful in a method for producing L-amino acid. The polynucleotide encodes a protein which is represented by SEQ ID NO:3 and has arginine at position 334 substituted by a terminator or encodes a protein which is represented by SEQ ID NO:31 and has tyrosine at position 592 substituted by phenylalanine, or is formed by mutations in bases at positions −45 bp and −47 bp of a promotor region represented by SEQ ID NO:57.

### CROSS-REFERENCE TO RELATED APPLICATION

The present application is a divisional application of and claims the priority benefit of a prior application Ser. No. 18/001,218, filed on Dec. 8, 2022. The prior application Ser. No. 18/001,218 is a 371 application of International PCT application serial no. PCT/CN2020/142133, filed on Dec. 31, 2020, which claimed the priorities of the Patent Application No. 202011105063.5 filed to State Intellectual Property Office of P.R.China on Oct. 15, 2020, entitled “A recombinant strain for producing L-amino acid, construction method therefor, and application thereof”; the patent application No. 202010790887.4 filed to State Intellectual Property Office of P.R.China on Aug. 7, 2020, entitled “A recombinant strain for producing L-amino acid, construction method therefor, and application thereof”; and the Patent Application No. 202010514037.1 filed to State Intellectual Property Office of P.R.China on Jun. 8, 2020, entitled “A recombinant strain for lysc gene modification, construction method therefor, and application thereof”. The three prior applications are all incorporated herein by reference in their entireties.

### REFERENCE TO SEQUENCE LISTING

A Sequence Listing submitted as an XML file via EFS-Web is hereby incorporated by reference in accordance with 35 U.S.C. § 1.52 (e). The name of the XML file for the Sequence Listing is 134711-US-Sequence-Listing, the date of creation of the XML file is Oct. 20, 2025, and the size of the XML file is 91,550 bytes.

### DEPOSIT OF MICROORGANISM

The following microorganism has been deposited in accordance with the terms of the Budapest Treaty with the China General Microbiological Culture Collection Center (CGMCC) on the date indicated:

| Microorganism | Accession No. | Date |
|---|---|---|
| Corynebacterium | CGMCC No. 12856 | Aug. 16, 2016 |
| glutamicum | | |

Corynebacterium glutamicum was deposited under Accession Number CGMCC No. 12856 on Aug. 16, 2016 with the China General Microbiological Culture Collection Center (CGMCC). This deposit was made under the provisions of the Budapest Treaty on the International Recognition of the Deposit of Microorganisms for the Purposes of Patent Procedure and the Regulations thereunder (Budapest Treaty). This assures maintenance of a viable culture of the deposit for 30 years from date of deposit. The deposit will be made available by CGMCC under the terms of the Budapest Treaty, and subject to an agreement between Applicant and CGMCC which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14). Availability of the deposited strain is not to be construed as a license to practice the invention in contravention of the rights granted under the authority of any government in accordance with its patent laws.

### TECHNICAL FIELD

The invention belongs to the field of genetic engineering and microbial technology, particularly relates to a recombinant strain producing L-amino acid, construction method therefor, and application thereof.

### BACKGROUND

L-lysine has a wide range of applications, including medicine, food, feed, and other aspects. Among them, L-Lysine used as feed additive accounts for more than 90% of the total amount. At present, China is the second largest consumer market and the largest producer for L-lysine.

At present, L-lysine is mainly produced by direct fermentation, which uses strains with complete biosynthetic pathway of L-lysine and takes waste molasses, starch hydrolysate and the like as substrates to produce L-lysine via aerobic fermentation. This method accounts for ⅔ of the production of L-lysine all over the world today, and its process is very mature. This method mainly exists in yeast, bacteria and mold, and widely exists in microorganisms. At present, the production strains used for L-lysine fermentation in industry are mainly mutagenesis breeding mutant strains of Corynebacterium and Brevibacterium genus. With the development of metabolic engineering and genetic engineering, gene mutation became controllable. Therefore, in the process of engineering a starting strain with metabolic engineering, it is possible to accurately find out the key enzyme genes for L-lysine production in the metabolic process, and then to improve the expression of such key enzyme genes, so that the increase in L-lysine production can be achieved.

L-glutamic acid is mainly used in the production of monosodium glutamate and spices, and used as a salt substitute, nutritional supplement and biochemical reagent and so on. L-glutamic acid itself can be used as a drug to participate in the metabolism of protein and sugar in the brain, so as to promote the oxidation process. This product combines with ammonia within the body to synthesize non-toxic glutamine, which can reduce blood ammonia and alleviate the symptoms of liver coma. In the past, the production of monosodium glutamate is mainly conducted by the hydrolysis of wheat gluten (glutenin), and now, the microbial fermentation method is used for large-scale production.

### SUMMARY OF INVENTION

A purpose of the present invention is to develop a new strain with the production capacity of L-amino acid, thereby to provide a method for effectively producing L-amino acid.

In order to achieve the above purpose, the inventor of the invention has found via research that NCgl0609 gene and/or NCgl1575 gene with amino acid production capacity via fermentation can have high efficient L-amino acid production capacity by modifying the gene or improving its expression, which is unknown in the prior art; in addition, the inventor of the invention also found that mutation of a certain promoter sequence can also improve the L-amino acid production capacity of corresponding microorganisms. Based on these findings, the invention is completed.

The invention provides a bacterium producing L-amino acids, wherein the expression of a polynucleotide encoding a amino acid sequence of SEQ ID NO: 3 is improved, and/or the expression of a polynucleotide encoding an amino acid sequence of SEQ ID NO: 31 is improved, and/or the bases at positions −45 bp and −47 bp of a promoter region shown in SEQ ID NO: 57 are mutated. The invention also provides a method for producing L-amino acid by using the microorganism.

According to the invention, the improvement in expression is that the expression of the polynucleotide is enhanced or the polynucleotide encoding an amino acid sequence of SEQ ID NO: 3 or SEQ ID NO: 31 has point mutations, or the polynucleotide encoding an amino acid sequence of SEQ ID NO: 3 or SEQ ID NO: 31 has point mutations and the expression is enhanced.

A first aspect of the invention provides a bacterium that produces L-amino acid, in which the expression of polynucleotide encoding the amino acid sequence of SEQ ID NO: 3 is improved. Preferably, the L-amino acid is L-lysine or L-glutamic acid.

The amino acid sequence of SEQ ID NO: 3 is a protein encoded by gene NCgl0609.

The bacterium has enhanced L-amino acid production capacity.

A bacterium with L-amino acid production capacity can be a bacterium that can accumulate the target L-amino acid in the culture medium in an amount of preferably more than 0.5 g/L, more preferably more than 1.0 g/L.

The polynucleotides can encode amino acid sequences with sequence homology of about 90% or more, about 92% or more, about 95% or more, about 97% or more, about 98% or more, or about 99% or more with the amino acid sequence of SEQ ID NO: 3.

In one specific embodiment of the invention, the polynucleotide with improved expression contains the nucleotide sequence of SEQ ID NO: 1.

In one embodiment of the invention, the improvement in expression means that the polynucleotide encoding the amino acid sequence of SEQ ID NO: 3 has point mutations, such that arginine at position 334 of the amino acid sequence of SEQ ID NO: 3 is substituted by a terminator.

According to the invention, the amino acid sequence in which arginine at position 334 of the amino acid sequence shown in SEQ ID NO: 3 is substituted by the terminator is shown in SEQ ID NO: 4.

In one embodiment of the invention, the polynucleotide sequence with point mutation is formed by mutation of the 1000^{th} base of the polynucleotide sequence shown in SEQ ID NO: 1.

According to the invention, the mutation includes the mutation of the 1000^{th} base of the polynucleotide sequence shown in SEQ ID NO: 1 from cytosine (C) to thymine (T).

In one embodiment of the invention, the polynucleotide sequence with point mutation includes the polynucleotide sequence shown in SEQ ID NO: 2.

The invention also provides a bacterium that produces L-amino acids, which have improved expression of polynucleotide encoding the amino acid sequence of SEQ ID NO: 31. Preferably, the L-amino acid is L-lysine. Preferably, the bacterium is a bacterium that belongs to Corynebacterium genus.

The amino acid sequence of SEQ ID NO: 31 is a protein encoded by gene NCgl1575.

The microorganism has enhanced L-lysine production capacity compared with a wild type or parent strain.

The polynucleotides can encode amino acid sequences with sequence homology of about 90% or more, about 92% or more, about 95% or more, about 97% or more, about 98% or more, or about 99% or more with the amino acid sequence of SEQ ID NO: 31.

In one specific embodiment of the invention, the polynucleotide can contain the nucleotide sequence of SEQ ID NO: 29.

In one embodiment of the invention, the polynucleotide encoding the amino acid sequence of SEQ ID NO: 31 has point mutations, such that tyrosine at position 592 of the amino acid sequence of SEQ ID NO: 31 is substituted by different amino acids.

According to the invention, it is preferred that tyrosine at position 592 is substituted by phenylalanine.

According to the invention, the amino acid sequence in which tyrosine (Y) at position 592 of the amino acid sequence shown in SEQ ID NO: 31 is substituted by phenylalanine (F) is shown in SEQ ID NO: 32.

In one embodiment of the invention, the polynucleotide sequence with point mutation is formed by mutation of the 1775^{th} base of the polynucleotide sequence shown in SEQ ID NO: 29.

According to the invention, the mutation includes the mutation of the 1775^{th} base of the polynucleotide sequence shown in SEQ ID NO: 29 from adenine (A) to thymine (T).

In one embodiment of the invention, the polynucleotide sequence with point mutation includes the polynucleotide sequence shown in SEQ ID NO: 30.

According to the invention, the bacterium can be a microorganism that belongs to Corynebacterium genus, such as Corynebacterium glutamicum, Brevibacterium flavum, Brevibacterium lactofermentum, Corynebacterium ammoniagenes, Corynebacterium pekinense.

In one embodiment of the invention, the microorganism belonging to Corynebacterium genus is Corynebacterium glutamicum YP97158, with the deposition number CGMCC No. 12856, deposited on Aug. 16, 2016, the depository unit is the General Microbiology Center of China Microbial Species Conservation and Management Commission, No. 3, Yard. 1 Beichen West Road, Chaoyang District, Beijing, Tel: 010-64807355, recorded in Chinese patent application CN106367432A (the filing date: Sep. 1, 2016, publication date: Feb. 1, 2017).

In one embodiment of the invention, the microorganism belonging to Corynebacterium genus is Corynebacterium glutamicum ATCC 13869.

The expression of polynucleotides can be enhanced by the following means: substitution or mutation in expression regulation sequences, introduction of mutation to polynucleotide sequences, increase of the number of copies of polynucleotides introduced through chromosome insertion or vector, or combinations thereof.

The expression regulation sequences of polynucleotides can be modified. The expression regulation sequences control the expression of polynucleotides to which they are operably linked, and may include promoters, terminators, enhancers, silencers, and the like, for example. Polynucleotides can have changes in the starting codon. Polynucleotides can be incorporated into specific sites of chromosomes so as to increase the number of copies. Herein, specific sites may include, for example, transposon sites or intergenic sites. In addition, polynucleotides can be incorporated into an expression vector, and the expression vector can be introduced into host cells to increase the number of copies.

In one embodiment of the invention, the number of copies is increased by incorporating polynucleotides or polynucleotides with point mutations to specific sites of microbial chromosomes.

In one embodiment of the invention, the nucleic acid sequence is overexpressed by incorporating polynucleotides with promoter sequences or polynucleotides with promoter sequences and point mutations into specific sites of microbial chromosomes.

In one embodiment of the invention, the number of copies is increased by incorporating polynucleotides or polynucleotides with point mutations into expression vectors, and introducing the expression vectors into host cells.

In one embodiment of the invention, the amino acid sequence is overexpressed by incorporating polynucleotides with promoter sequences or the polynucleotides with promoter sequences and point mutations into expression vectors, and introducing the expression vectors into host cells.

In one specific embodiment of the invention, the promoter is the promoter of polynucleotide (NCgl0609 gene) encoding the amino acid sequence of SEQ ID NO: 3.

In one specific embodiment of the invention, the promoter is the promoter of polynucleotide (NCgl1575 gene) encoding the amino acid sequence of SEQ ID NO: 31.

In some specific embodiments of the invention, the vectors used are pK18mobsacB plasmid and pXMJ19 plasmid.

According to the invention, the bacterium can also have other improvements associated with increase in the production of L-amino acids.

The second aspect of the invention provides a polynucleotide sequence, an amino acid sequence encoded by the polynucleotide sequence, a recombinant vector including the polynucleotide sequence, and a recombinant strain containing the polynucleotide sequence.

According to the invention, the polynucleotide sequence has improved expression, and the improvement includes point mutations of polynucleotide encoding polypeptide containing amino acid sequence shown in SEQ ID NO: 3, such that arginine at 334 position of the amino acid sequence is substituted by a terminator.

According to the invention, the amino acid sequence in which arginine at 334 position of the amino acid sequence shown in SEQ ID NO: 3 is substituted by a terminator is shown in SEQ ID NO: 4.

According to the invention, the polynucleotide sequence encoding the polypeptide containing the amino acid sequence shown in SEQ ID NO: 3 contains the polynucleotide sequence shown in SEQ ID NO: 1.

In one embodiment of the invention, the mutated polynucleotide sequence provided by the invention is formed by mutation of the 1000^{th} base of the polynucleotide sequence shown in SEQ ID NO: 1.

According to the invention, the mutation includes the mutation of the 1000^{th} base of the polynucleotide sequence shown in SEQ ID NO: 1 from cytosine (C) to thymine (T).

In one embodiment of the invention, the mutated polynucleotide sequence includes the polynucleotide sequence shown in SEQ ID NO: 2.

According to the invention, the substituted amino acid sequence includes the amino acid sequence shown in SEQ ID NO: 4.

According to the invention, the polynucleotide sequence includes polynucleotide encoding polypeptides containing amino acid sequence shown in SEQ ID NO: 31, wherein tyrosine at position 592 is substituted by different amino acids.

According to the invention, preferably, tyrosine at position 592 is substituted by phenylalanine.

According to the invention, the amino acid sequence in which tyrosine (Y) at position 592 of the amino acid sequence shown in SEQ ID NO: 31 is substituted by phenylalanine (F) is shown in SEQ ID NO: 32.

According to the invention, preferably, the polynucleotide sequence encoding the polypeptide containing the amino acid sequence shown in SEQ ID NO: 31 contains the polynucleotide sequence shown in SEQ ID NO: 29.

In one embodiment of the invention, the polynucleotide sequence is formed by mutation of the 1775^{th} base of the polynucleotide sequence shown in SEQ ID NO: 29.

According to the invention, the mutation includes the mutation of the 1775^{th} base of the polynucleotide sequence shown in SEQ ID NO: 29 from adenine (A) to thymine (T).

In one embodiment of the invention, the polynucleotide sequence includes the polynucleotide sequence shown in SEQ ID NO: 30.

According to the invention, the amino acid sequence includes the amino acid sequence shown in SEQ ID NO: 32.

According to the invention, the mutation refers to change of the base/nucleotide of the site. The mutation method can be selected from at least one of mutagenesis, PCR site directed mutation method, and/or homologous recombination method. In the invention, PCR site directed mutation method and/or homologous recombination method are preferred.

According to the invention, the recombinant vector is constructed by introducing the polynucleotide sequence into the plasmid.

In one embodiment of the invention, the plasmid is pK18mobsacB plasmid.

In another embodiment of the invention, the plasmid is pXMJ19 plasmid.

Specifically, the polynucleotide sequence and the plasmid can be constructed into a recombinant vector via a NEBuider recombination system.

According to the invention, the recombinant strain contains the polynucleotide sequence.

As one embodiment of the invention, the starting strain of the recombinant strain is YP97158.

As one embodiment of the invention, the starting strain of the recombinant strain is ATCC 13869.

The third aspect of the invention also provides a method for constructing a recombinant strain for producing L-amino acid.

According to the invention, the construction method includes the following steps:

Modifying the polynucleotide sequence of wild-type NCgl0609 shown in SEQ ID NO: 1 in a host strain to mutate its 1000^{th} base, so as to obtain a recombinant strain containing the mutated NCgl0609 encoding gene.

According to the construction method of the invention, the modification includes at least one of mutagenesis, PCR site directed mutation method, and/or homologous recombination method.

According to the construction method of the invention, the mutation refers to the mutation of the 1000^{th} base in SEQ ID NO: 1 from cytosine (C) to thymine (T); Specifically, the polynucleotide sequence containing the mutated NCgl0609 encoding gene is shown in SEQ ID NO: 2.

Further, the construction method comprises the following steps:
(1) Modifying the nucleotide sequence of wild-type NCgl0609 gene as shown in SEQ ID NO: 1 to mutate its 1000^{th} base, so as to obtain the mutated polynucleotide sequence of NCgl0609 gene;
(2) Linking the mutated polynucleic acid sequence with a plasmid to construct a recombinant vector; and
(3) Introducing the recombinant vector into a host strain to obtain the recombinant strain containing the mutated NCgl0609 encoding gene.

According to the construction method of the invention, the step (1) includes: constructing the NCgl0609 gene with point mutation: synthesizing two pairs of primers P1 and P2, P3 and P4 for amplifying NCgl0609 gene fragments based on the genome sequence of the unmodified strain, and introducing point mutation into SEQ ID NO: 1 of wild-type NCgl0609 gene by PCR site directed mutation method, to obtain the nucleotide sequence SEQ ID NO: 2 of NCgl0609 gene with point mutation, which is recorded as NCgl0609^{C1000T}.

In one embodiment of the invention, the genome of the unmodified strain can be derived from ATCC13032 strain, and its genome sequence can be obtained from NCBI website.

In one embodiment of the invention, in the step (1), the primers are:

P1:
(SEQ ID NO: 5)
5′CAGTGCCAAGCTTGCATGCCTGCAGGTCGACTCTAGG
GACGGCAACGTACATAAC3′,
P2:
(SEQ ID NO: 6)
5′ GTTGCCGGTGAGTCAAACAGTCATTTTGC 3′,
P3:
(SEQ ID NO: 7)
5′ GCAAAATGACTGTTTGACTCACCGGCAAC 3′,
and
P4:
(SEQ ID NO: 8)
5′ CAGCTATGACCATGATTACGAATTCGAGCTCGGTACC
CGCGGCTG GAAATGTGGAG3′.

In one embodiment of the invention, the PCR amplification is carried out as follows: pre-denaturation for 5 min at 94° C., denaturation for 30 s at 94° C., annealing for 30 s at 52° C., extension for 40 s at 72° C. (30 cycles), and over extension for 10 min at 72° C.

In one embodiment of the invention, the overlap PCR amplification is carried out as follows: pre-denaturation for 5 min at 94° C., denaturation for 30 s at 94° C., annealing for 30 s at 52° C., extension for 60 s at 72° C. (30 cycles), and over extension for 10 min at 72° C.

According to the construction method of the invention, the step (2) includes the construction of a recombinant plasmid, including: assembling the separated and purified NCgl0609^{C1000T} and pK18mobsacB plasmids through a NEBuider recombination system to obtain the recombinant plasmid.

According to the construction method of the invention, the step (3) includes construction of a recombinant strain: transforming the recombinant plasmid into a host strain, to obtain the recombinant strain.

In one embodiment of the invention, the transforming in the step (3) is an electric transforming method.

In one embodiment of the invention, the host strain is YP97158.

In one embodiment of the invention, the recombination is achieved by homologous recombination.

The fourth aspect of the invention also provides a method for constructing a recombinant strain for producing L-amino acid.

According to the invention, the construction method includes the following steps: Amplifying the upstream and downstream homologous arm fragments of NCgl0609 gene, the coding region of NCgl0609 gene and its promoter region sequence, or amplifying the coding region of NCgl0609 or NCgl0609^{R334*} gene and its promoter region sequence, and then introducing NCgl0609 or NCgl0609^{R334*} gene into the genome of a host strain by homologous recombination, so as to obtain the overexpression of NCgl0609 or NCgl0609^{R334*} gene in the strain.

In one embodiment of the invention, the primers for amplifying the upstream homologous arm fragment are:

P7:
(SEQ ID NO: 11)
5′CAGTGCCAAGCTTGCATGCCTGCAGGTCGACTCTAG
AATGCGTTCTG GACTGAGG 3′,
and
P8:
(SEQ ID NO: 12)
5′ GAGATGATCCTCGCAGCTGGTGCACCGAGAACAGATG 3′.

In one embodiment of the invention, the primers for amplifying the downstream homologous arm fragment are:

P11:
(SEQ ID NO: 15)
5′ GGTCAAGGAAGGAGTTGTTGCCAGAATCAGATG
GCGCAATTA AATC AAG 3′,
and
P12:
(SEQ ID NO: 16)
5′ CAGCTATGACCATGATTACGAATTCGAGCTCGGTA
CCCGCTATGACACCTTCAACGGATC 3′.

In one embodiment of the invention, the primers for amplifying the sequence of the gene coding region and its promoter region are:

P9:
(SEQ ID NO: 13)
5′ CATCTGTTCTCGGTGCACCAGCTGCGAGGATCATCTC 3′,
and
P10:
(SEQ ID NO: 14)
5′ GATTTAATTGCGCCATCTGATTCTGGCAACAACTCC
TTCCTTGACC 3′.

In one embodiment of the invention, the above P7-P12 are used as primers, and the upstream homologous fragment, downstream homologous fragment and NCgl0609 or NCgl0609^{R334*} fragment with its own promoter obtained through amplification are mixed as templates for amplification, so as to obtain an integrated homologous arm fragment.

In one embodiment of the invention, PCR system used is: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume: 50 μL; the PCR amplification is carried out as follows: pre-denaturation for 5 min at 94° C., denaturation for 30 s at 94° C., annealing for 30 s at 52° C., extension for 60 s at 72° C. (30 cycles), and over extension for 10 min at 72° C.

In one embodiment of the invention, a shuttle plasmid PK18mobsacB is assembled with upper and lower homologous arm fragments, gene coding region and promoter region fragments, by using a NEBuider recombination system, so as to obtain the integrated plasmid.

In one embodiment of the invention, the integrated plasmid is transfected into the host strain, and NCgl0609 or NCgl0609^{R334*} gene is introduced into the genome of the host strain by homologous recombination.

In one embodiment of the invention, the host strain is YP97158.

In one embodiment of the invention, the host strain is a strain carrying the polynucleotide sequence shown in SEQ ID NO: 2.

The fifth aspect of the invention also provides a method for constructing a recombinant strain for producing L-amino acid.

According to the invention, the construction method includes the following steps: Amplifying the NCgl0609 gene coding region and promoter region sequence, or the NCgl0609^{R334}*gene coding region and promoter region sequence, constructing an overexpression plasmid vector, and transferring the vector into a host strain to achieve overexpression of NCgl0609 or NCgl0609^{R334*} in the strain.

In one embodiment of the invention, the primers for amplifying the sequence of the gene coding region and its promoter region are:

P17:
(SEQ ID NO: 21)
5′GCTTGCATGCCTGCAGGTCGACTCTAGAGGATCCCCCAGC
TGCGAGG A TCATCTC3′,
and
P18:
(SEQ ID NO: 22)
5′ATCAGGCTGAAAATCTTCTCTCATCCGCCAAAACCAACA
ACTCCTTCCTTGACC3′.

In one embodiment of the invention, the PCR system is: 10×Ex Taq Buffer 5 μL, dNTP Mixture (each 2.5 mM) 4 μL, Mg²⁺ (25 mM) 4 μL, primer (10 μM) each 2 μL, Ex Taq (5 U/μL) 0.25 μL, total volume 50 μL; the PCR amplification is carried out as follows: pre-denaturation for 5 min at 94° C., denaturation for 30 s at 94° C., annealing for 30 s at 52° C., extension for 60 s at 72° C. (30 cycles), and over extension for 10 min at 72° C.

In one embodiment of the invention, a shuttle plasmid pXMJ19 is assembled with NCgl0609 and NCgl0609^{R334*} fragments with their own promoters by using a NEBuider recombination system, so as to obtain the overexpression plasmid.

In one embodiment of the invention, the host strain is YP97158.

In one embodiment of the invention, the host strain is ATCC 13869.

In one embodiment of the invention, the host strain is a strain carrying the polynucleotide sequence shown in SEQ ID NO: 2.

The invention also provides a method for constructing a corynebacterium recombinant strain.

According to the invention, the construction method includes the following steps: Modifying the polynucleotide sequence of wild-type NCgl1575 in the host strain as shown in SEQ ID NO: 29 to mutate its 1775^{th} base, so as to obtain a Corynebacterium recombinant strain containing the mutated NCgl1575 encoding gene.

According to the construction method of the invention, the modification includes at least one of mutagenesis, PCR site directed mutation method, and/or homologous recombination.

According to the construction method of the invention, the mutation refers to the mutation of the 1775^{th} base in SEQ ID NO: 29 from adenine (A) to thymine (T); Specifically, the polynucleotide sequence containing the mutated NCgl1575 encoding gene is shown in SEQ ID NO: 30.

Further, the construction method comprises the following steps:
(1) Modifying the nucleotide sequence of wild-type NCgl1575 gene shown in SEQ ID NO: 29 to mutate its 1775^{th} base, so as to obtain the mutated polynucleotide sequence of NCgl1575 gene;
(2) Linking the mutated polynucleic acid sequence with a plasmid to construct a recombinant vector; and
(3) Introducing the recombinant vector into a host strain to obtain the Corynebacterium recombinant strain containing the mutated NCgl1575 encoding gene.

According to the construction method of the invention, the step (1) includes: constructing the NCgl1575 gene with point mutation: synthesizing two pairs of primers P1′ and P2′, and P3′ and P4′ for amplifying NCgl1575 gene fragments, based on the genome sequence of Corynebacterium glutamicum, and introducing point mutation into SEQ ID NO: 29 of wild-type NCgl1575 gene by PCR site directed mutation method, to obtain the nucleotide sequence SEQ ID NO: 30 of NCgl1575 gene with point mutation, which is recorded as NCgl1575^{A1775T}.

In one embodiment of the invention, the genome of Corynebacterium glutamicum can be derived from ATCC13032 strain, and its genome sequence can be obtained from NCBI website.

In one embodiment of the invention, in the step (1), the primers are:

P1′:
(SEQ ID NO: 33)
5′CAGTGCCAAGCTTGCATGCCTGCAGGTCGACTCTAGTGCGTT
CGTCTGCGGTTTCG 3′;
P2′:
(SEQ ID NO: 34)
5′ ATCGACGCCGCCCCATTCACCCTTCTGATG 3′;
P3′:
(SEQ ID NO: 35)
5′ CATCAGAAGGGTGAATGGGGCGGCGTCGAT 3′;
and
P4′:
(SEQ ID NO: 36)
5′CAGCTATGACCATGATTACGAATTCGAGCTCGGTACCC
AAGCCTCGACCCCTACATC 3′.

In one embodiment of the invention, the PCR amplification is carried out as follows: denaturation for 30 s at 94° C., annealing for 30 s at 52° C., and extension for 40 s at 72° C. (30 cycles).

In one embodiment of the invention, the overlap PCR amplification is carried out as follows: denaturation for 30 s at 94° C., annealing for 30 s at 52° C., and extension for 90 s at 72° C. (30 cycles).

According to the construction method of the invention, the step (2) includes the construction of a recombinant plasmid, including: assembling the separated and purified NCgl1575^{A1775T} and pK18mobsacB plasmids through a NEBuider recombination system to obtain the recombinant plasmid pK18-NCgl1575^{A1775T}.

According to the construction method of the invention, the step (3) includes construction of a recombinant strain: transforming the recombinant plasmid pK18-NCgl1575^{A1775T} into a host strain, to obtain the recombinant strain.

In one embodiment of the invention, the transforming in the step (3) is an electric transforming method.

In one embodiment of the invention, the host strain is YP97158.

In one embodiment of the invention, the recombination is achieved by homologous recombination.

The invention also provides a method for constructing a corynebacterium recombinant strain.

According to the invention, the construction method includes the following steps:

Amplifying the upstream and downstream homologous arm fragments of NCgl1575 gene, the coding region of NCgl1575 gene and its promoter region sequence, or the coding region of NCgl1575^{A1775T} gene and its promoter region sequence, and introducing NCgl1575 or NCgl1575^{A1775T} gene into the genome of a host strain by homologous recombination, so as to achieve overexpression of NCgl1575 or NCgl1575^{A1775T} gene in the strain.

In one embodiment of the invention, the primers for amplifying the upstream homologous arm fragment are:

P7′:
(SEQ ID NO: 39)
5′ CAGTGCCAAGCTTGCATGCCTGCAGGTCGACTCTAGAATG
CGTTCTGGACTGAGG 3′;
and
P8′:
(SEQ ID NO: 40)
5′ GAAACGGCCTTAAGCTAGGTGCACCGAG AACAGATG 3′.

In one embodiment of the invention, the primers for amplifying the downstream homologous arm fragment are:

P11′:
(SEQ ID NO: 43)
5′ AACCGGGCGG GAAAAGCTTGATGGCGCAATTAAATCAAG 3′;
and
P12′:
(SEQ ID NO: 44)
5′CAGCTATGACCATGATTACGAATTCGAGCTCGGTACCC GCTAT
GACACCTTCAACGGATC 3′.

and

In one embodiment of the invention, the primers for amplifying the sequence of the gene coding region and its promoter region are:

P9′:
(SEQ ID NO: 41)
5′ CATCTGTTCTCGGTGCAC CTAGCTTAAG GCCGTTTC 3′;
and
P10′:
(SEQ ID NO: 42)
5′ CTTGATTTAATTGCGCCATCAAGCTTTTCC CGCCCGGTT 3′.

In one embodiment of the invention, the above P7′/P12′ are used as primers, and the upstream homologous fragment, downstream homologous fragment and NCgl1575 or NCgl1575^{A1775T} fragment with its own promoter obtained through amplification are mixed as templates for amplification, so as to obtain an integrated homologous arm fragment.

In one embodiment of the invention, PCR system used is: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL; PCR amplification is carried out as follows: pre-denaturation for 5 min at 94° C., denaturation for 30 s at 94° C., annealing for 30 s at 52° C., extension for 180 s at 72° C. (30 cycles), and over extension for 10 min at 72° C.

In one embodiment of the invention, a shuttle plasmid PK18mobsacB is assembled with the intergrated homologous arm fragment, by using a NEBuider recombination system, so as to obtain the integrated plasmid.

In one embodiment of the invention, the integrated plasmid is transfected into the host strain, and NCgl1575 or NCgl1575^{A1775T} gene is introduced into the genome of the host strain by homologous recombination.

In one embodiment of the invention, the host strain is YP97158.

In one embodiment of the invention, the host strain is a strain carrying the polynucleotide sequence shown in SEQ ID NO: 30.

The invention also provides a method for constructing a Corynebacterium recombinant strain.

According to the invention, the construction method includes the following steps:

Amplifying the NCgl1575 gene coding region and promoter region sequence, or the NCgl1575^{A1775T} gene coding region and promoter region sequence, constructing an overexpression plasmid vector, and transferring the vector into a host strain to achieve overexpression of NCgl1575 or NCgl1575^{A1775T} gene in the strain.

In one embodiment of the invention, the primers for amplifying the sequence of the gene coding region and its promoter region are:

P17′:
(SEQ ID NO: 49)
5′GCTTGCATGCCTGCAGGTCGACTCTAGAGGATCCCC CTAGCT
TAAG GCCGTTTC 3′;
and
P18′:
(SEQ ID NO: 50)
5′ATCAGGCTGAAAATCTTCTCTCATCCGCCAAAAC AAGCTTT
TCC CGCCCGGTT 3′.

In one embodiment of the invention, the PCR system is: 10×Ex Taq Buffer 5 μL, dNTP Mixture (each 2.5 mM) 4 μL, Mg²⁺(25 mM) 4 μL, primers (10 μM) each 2 μL, Ex Taq (5 U/μL) 0.25 μL, total volume 50 μL; the PCR amplification is carried out as follows: pre-denaturation for 5 min at 94° C., denaturation for 30 s at 94° C., annealing for 30 s at 52° C., extension for 120 s at 72° C. (30 cycles), and over extension for 10 min at 72° C.

In one embodiment of the invention, a shuttle plasmid pXMJ19 is assembled with NCgl1575 or NCgl1575^{A1775T} fragments with their own promoters by using a NEBuider recombination system, so as to obtain the overexpression plasmid.

In one embodiment of the invention, the host strain is YP97158.

In one embodiment of the invention, the host strain is a strain carrying the polynucleotide sequence shown in SEQ ID NO: 30.

Another aspect of the invention is to provide a promoter nucleotide sequence, which includes the nucleotide sequence formed by mutation of bases at positions −45 bp and −47 bp in the promoter region shown in SEQ ID NO: 57.

According to the invention, nucleotide guanine (G) at position −45 bp is mutated to adenine (A), and nucleotide guanine (G) at position −47 bp is mutated to thymine (T) in the promoter region shown in SEQ ID NO: 57.

According to the invention, the promoter nucleotide sequence is as follows:
(a) the nucleotide sequence shown in SEQ ID NO: 58; or,
(b) the nucleotide sequence having a sequence identity of more than 90%, preferably more than 95%, 98% to nucleotide sequence shown in SEQ ID NO: 58, and retaining enhanced activity of the promoter of (a), with at position −45 bp remaining as adenine (A), and at position −47 bp remaining as thymine (T).

The invention also provides an expression cassette containing the above promoter, including the promoter and an encoding sequence that can be operatively connected behind the promoter. In one embodiment of the invention, the encoding sequence is the encoding sequence of lysC gene.

The invention also provides a recombinant vector containing the promoter nucleotide sequence of the invention.

According to the invention, the recombinant vector is constructed by linking the promoter nucleotide sequence of the invention with a shuttle plasmid; as an embodiment of the invention, the shuttle plasmid is pK18mobsacB plasmid.

The invention also provides a recombinant strain comprising the promoter nucleotide sequence or the recombinant vector above.

The recombinant strain according to the invention comprises the nucleotide sequence shown in SEQ ID NO: 58. The nucleotide sequence shown in SEQ ID NO: 58 is the promoter region of lysC gene. Further, the nucleotide sequence shown in SEQ ID NO: 58 is linked with the lysC gene encoding sequence. In particular, the recombinant strain can include expression the cassette or the recombinant vector as described above in the invention. In particular, the recombinant strain of the invention is obtained by transforming the expression cassette or the recombinant vector. The recombinant strain according to the invention is formed by introducing the nucleotide sequence of the mutated promoter above into a host strain for recombination; the host strain can be selected from strains that produce L-amino acid, especially L-lysine, as known in the art, for example, at least one selected from Corynebacterium. The Corynebacterium can be Corynebacterium glutamicum, Corynebacterium flavum, Corynebacterium crenatum and Corynebacterium pekinene; Corynebacterium glutamicum is preferred. As an embodiment of the invention, the host strain is YP97158.

The recombinant strain according to the invention uses the pK18mobsacB plasmid as the vector.

The recombinant strain according to the invention may further include other modifications.

The invention also provides a method for constructing a recombinant strain producing L-lysine, which comprises the following step:
(1) Modifying the promoter region shown in SEQ ID NO:57, to mutate position −45 bp and −47 bp, so as to obtain the nucleotide sequence containing the mutated promoter region.

According to the invention, the mutation refers to the mutation of nucleotide guanine (g) at position −45 bp to adenine (a) and nucleotide guanine (g) at position −47 bp to thymine (T) in the promoter region shown in SEQ ID NO: 57. Specifically, the nucleotide sequence of the mutated promoter region is shown in SEQ ID NO: 58. Further, the construction method further comprises the following steps:
(2) Linking the nucleotide sequence of the mutated promoter region with a plasmid to construct a recombinant vector; and
(3) Introducing the recombinant vector into a host strain to obtain the recombinant strain producing L-lysine containing the mutated promoter region.

According to the invention, the method of mutation in step (1) includes mutagenesis, PCR site directed mutation or homologous recombination, preferably PCR site directed mutation.

According to the invention, the step (1) comprises:
Designing two pairs of primers to amplify the promoter region of lysc gene, and then obtaining the nucleotide sequence of the mutated promoter region by PCR technology.

In an embodiment of the present invention, the primers in step (1) are:

P1″:
(SEQ ID NO: 59)
5′ CCGGAATTCG ACCAAGGATG AGGGCTTTG 3′(EcoR I);
P2″:
(SEQ ID NO: 60)
5′ AGTTACCCGC TCAATTATAC CTTTATAAAC 3′;
P3″:
(SEQ ID NO: 61)
5′ GTTTATAAAG GTATAATTGA GCGGGTAACT 3′;
and
P4″:
(SEQ ID NO: 62)
5′ ACATGCATGC GCGTACGCGA AGTGGCACAT 3′(Sph I).

In one embodiment of the present invention, the step (1) includes: using Corynebacterium glutamicum ATCC13032 as a template and using primers P1″ and P2″, P3″ and P4″, respectively, to perform PCR amplification to obtain two DNA fragments; Using the above two DNA fragments as templates and P1″ and P4″ as primers, to obtain the DNA fragment containing the promoter region nucleotide sequence (SEQ ID NO: 58) of the present invention by overlap PCR amplification.

According to the invention, in step (1), the DNA fragment obtained through overlap PCR amplification contains EcoR I and Sph I enzyme digestion sites at both ends, respectively.

According to the invention, the step (2) includes: subjecting the product amplified through overlap PCR reaction to agarose gel electrophoresis and separation and purification, connecting the fragment via double enzyme digestion (EcoR I Sph I) with a shuttle plasmid avia the same double enzyme digestion ((EcoR I Sph I)), to obtain an allelic replaced recombinant vector.

According to the invention, the shuttle plasmid is a pK18mobsacB plasmid; and the constructed recombinant vector is pK18-Plys C^{(G(-45)A,G(-47)T)}.

In one embodiment of the present invention, the recombinant plasmid has a kanamycin resistance marker.

In one embodiment of the present invention, the transformation of step (3) is an electric transformation method; exemplary, in step (3), the recombinant vector is transformed into the strain YP97158.

The above various recombinant strains obtained by the invention can be used in fermentation to produce L-amino acids alone or in combination, or can be mixed with other bacterium producing L-amino acids for fermentation, so as to produce L-amino acids.

Another aspect of the invention provides a method for producing L-amino acids, which includes culturing the bacterium; and obtaining L-amino acids from the culture.

The bacterium can be cultured in a suitable medium under culture conditions known in the art. The culture medium can contain a carbon source, a nitrogen source, trace elements, and their combinations. In culture, the pH of the culture can be adjusted. In addition, in culture, the prevention of bubble generation can be included, for example, by using defoamers to prevent bubble from generating. In addition, in culture, the injection of gas into the culture can be included. Gases may include any gas capable of maintaining the aerobic conditions of the culture. In culture, the temperature of the culture can be 20 to 45° C. The generated L-amino acids can be recovered from the culture, that is, the culture is treated with sulfuric acid or hydrochloric acid, etc., followed by a combination of methods such as anion exchange chromatography, concentration, crystallization, and isoelectric point precipitation.

In the invention:

SEQ ID NO: 1: NCgl0609 wild-type ORF sequence:
GTGTCACACACCGCGTCCACACCGACGCCAGAGGAATACTCCGC
GCAGCAACCCAGCACCCAGGGCACTCGCGTTGAGTTCCGCGGCA
TAACCAAAGTCTTTAGCAACAATAAATCTGCTAAAACCACCGC
GCTTGATAATGTCACTCTCACCGTAGAACCCGGTGAGGTAATCG
GCATCATCGGTTACTCTGGCGCCGGCAAGTCCACTCTTGTCCGC
CTCATCAATGG
CCTTGACTCCCCCACGAGCGGTTCGTTGCTGCTCAACGGCACCG
ACATCGTCGGAATGCCCGAGTCTAAGCTGCGTAAACTGCGCAGT
AATATCGGCATGATTTTCCAGCAGTTCAACCTGTTCCAGTCGCG
TACTGCGGCTGGAAATGTGGAGTACCCGCTGGAAGTTGCCAAGA
TGGACAAGGCAGCTCGTAAAGCTCGCGTGCAAGAAATGCTCGAG
TTCGTCGGCCTGGGCGACAAAGGCAAAAACTACCCCGAGCAGCT
GTCGGGCGGCCAGAAGCAGCGCGTCG
GCATTGCCCGTGCACTGGCCACCAATCCAACGCTTTTGCTTGCC
GACGAAGCCACCTCCGCTTTGGACCCAGAAACCACCCATGAAGT
TCTGGAGCTGCTGCGCAAGGTAAACCGCGAACTGGGCATCACCA
TCGTTGTGATCACCCACGAAATGGAAGTTGTGCGTTCCATCGCA
GACAAGGTTGCTGTGATGGAATCCGGCAAAGTTGTGGAATACGG
CAGCGTCTACGAGGTGTTCT CCAATCCACA AACACAGGTTGC
TCAAAAGT TCGTGGCCAC CGCGCTGCGT
AACACCCCAGACCAAGTGGAATCGGAAGATCTGCTTAGCCATGA
GGGACGTCTGTTCACCATTGATCTGACTGAAACGTCCGGCTTCT
TTGCAGCAACCGCTCGTGCTGCCGAACAAGGTGCTTTTGTCAAC
ATCGTTCACGGTGGCGTGACCACCTTGCAACGCCAATCATTTGG
CAAAATGACTGTTCGACTCACCGGCAACACCGCTGCGATTGAAG
AGTTCTATCAAACCTTGACC AAGACCACGA CCATCAAGGA G
ATCACCCGATGA
SEQ ID NO: 2: NCgl0609^{R334*} ORF sequence:
GTGTCACACA CCGCGTCCAC ACCGACGCCA GAGGAATACT
CCGCGCAGCAACCCAGCACCCAGGGCACTCGCGTTGAGTTCCGC
GGCATAACCAAAGTCTTTAGCAACAATAAATCTGCTAAAACCAC
CGCGCTTGATAATGTCACTCTCACCGTAGAACCCGGTGAGGTAA
TCGGCATCATCGGTTACTCTGGCGCCGGCAAGTCCACTCTTGTC
CGCCTCATCAATGGCCTTGACTCCCCCACGAGCGGTTCGTTGCT
GCTCAACGGCACCGACATCGTCGGAATGCCCGAGTCTAAGCTGC
GTAAACTGCGCAGTAATATCGGCATGATTTTCCAGCAGTTCAAC
CTGTTCCAGTCGCGTACTGCGGCTGGAAATGTGGAGTACCCGCT
GGAAGTTGCCAAGATGGACAAGGCAGCTCGTAAAGCTCGCGTGC
AAGAAATGCTCGAGTTCGTCGGCCTGGGCGACAAAGGCAAAAAC
TACCCCGAGCAGCTGTCGGGCGGCCAGAAGCAGCGCGTCGGCAT
TGCCCGTGCACTGGCCACCAATCCAACGCTTTTGCTTGCCGACG
AAGCCACCTCCGCTTTGGACCCAGAAACCACCCATGAAGTTCTG
GAGCTGCTGCGCAAGGTAAACCGCGAACTGGGCATCACCATCGT
TGTGATCACCCACGAAATGGAAGTTGTGCGTTCCATCGCAGACA
AGGTTGCTGTGATGGAATCCGGCAAAGTTGTGGAATACGGCAGC
GTCTACGAGGTGTTCTCCAATCCACAAACACAGGTTGCTCAAAA
GTTCGTGGCCACCGCGCTGCGTAACACCCCAGACCAAGTGGAAT
CGGAAGATCTGCTTAGCCATGAGGGACGTCTGTTCACCATTGAT
CTGACTGAAACGTCCGGCTTCTTTGCAGCAACCGCTCGTGCTGC
CGAACAAGGTGCTTTTGTCAACATCGTTCACGGTGGCGTGACCA
CCTTGCAACGCCAATCATTTGGCAAAATGACTGTTTGACTCACC
GGCAACACCGCTGCGATTGAAGAGTTCTATCA AACCTTGACC
AAGACCACGA CCATCAAGGA GATCACCCGATGA
SEQ ID NO: 3: NCgl0609 wild-type coding
protein amino acid sequence:
MSHTASTPTPEEYSAQQPSTQGTRVEFRGITKVFSNNKSAKTTA
LDNVTLTVEPGEVIGIIGYSGAGKSTLVRLINGLDSPTSGSLLL
NGTDIVGMPESKLRKLRSNIGMIFQQFNLFQSRTAAGNVEYPLE
VAKMDKAARKARVQEMLEFVGLGDKGKNYPEQLSGGQKQRVGIA
RALATNPTLLLADEATSALDPETTHEVLELLRKVNRELGITIVV
ITHEMEVVRSIADKVAVMESGKVVEYGSVYEVFSNPQTQVAQKF
VATALRNTPDQVESEDLLSHEGRLFTIDLTETSGFFAATARAAE
QG AFVNIVHGGV TTLQRQSFGK MTVRLTGNTA AIEEFYQT
LT KTTTIKEITR
SEQ ID NO: 4: NCgl0609R334* coding protein
amino acid sequence:
MSHTASTPTPEEYSAQQPSTQGTRVEFRGITKVFSNNKSAKTTA
LDNVTLTVEPGEVIGIIGYSGAGKSTLVRLINGLDSPTSGSLLL
NGTDIVGMPESKLRKLRSNIGMIFQQFNLFQSRTAAGNVEYPLE
VAKMDKAARKARVQEMLEFVGLGDKGKNYPEQLSGGQKQRVGIA
RALATNPTLLLADEATSALDPETTHEVLELLRKVNRELGITIVV
ITHEMEVVRSIADKVAVMESGKVVEYGSVYEVFSNPQTQVAQKF
VATALRNTPDQVESEDLLSHEGRLFTIDLTETSGFFAATARAAE
QG AFVNIVHGGV TTLQRQSFGK MTV
SEQ ID NO: 29: NCgl1575 wild-type ORF sequence:
ATGGCAGAATCAAACGCTATGGACCGGGCACAAATCTCTGCACT
GCTAGATAGAGCACAGCACACAATCAACCTTGCCGAACAAGCAA
ACAACGTGCTCCGACTGTTGAAAACACCCGGAACGGCCACAGTA
GGGGACAACGGGACACTCGGCACCGATACCTATCTGATCCCATC
CCGCAACATCACCTGGCCTGACAACCTGTATGTCAACGTCTTTC
TAGACGGCATGAATGCAGAAGCCACCCTTACCGATTACGTCGCA
TCAGTCGCTTCGATCCCACGCCTATGCCAGATCATCAACGAGGG
CCAAGGCGGCATGTTCCGCAGACTATTCAACCCCACCAAGGTCC
AAGCCGGCGACCAAGCTGTCTTCGACCTCATGGTCAAACTCGAC
GAGATTTCATCTACCACCCACGAAGTCTCCCGCATGCTCGAGGG
CGTCCACGCTGCCCGCACCCGCCAACAACAAGGCGTTGCACTTT
TCCCAGGTATTCATGGAGTGGGAGAGCGCTACATCGAACGCGCA
CAACAGGTACTCGCCTCAGCCCTCGGTATCGCTGGATTCGGTGC
CGAACCCTGGGACGGACATACCCTTGCCCAAGCGCGCCGGGTAG
TCCAACGCTACGCCCAAGATCCTAACTCCGAATACCGGCTGAAA
AGCGAAGCCGAGAAACACCTCACATCCATCAACGAGCTCCGCGT
ACAGATACTCCTCGAACAACTCCCCGTTGATGCCCTACGCATGG
CTACCGACCACCGCCTGCGCTTTGGATCCCTCGATTCCATCCAC
GTCGCAACCGTCGCCGACGTCCTAAAAACACACACCTCCATCCT
CACCACCGTGCAAGGTATCGGCGCCCAAACCGCGGGGCGGATGA
AAGCCGCAGCAGAAACACTCAAACAAGAAGCACTACGCCGCCAA
AACACCTCCATCGGCGACGAACCTACCCAACCCGCCATGCGTCT
AATCAACGTGCTGGCCCGCTTCGACCAAACCGAAACCATCACGC
CCGAAGAACGCGCCCGCCGCACCCGCGTCATCGACTACGTAGAA
CACATACCCCCAAGCCTCGACCCCTACATCGTCATCAACCCAGC
AACGCCTGAGTTCAACAACTTCACCGACGACCTCCGCTGGATCG
ACGCAA ACCCCAACCTCTTCCACCCACAAACAATCACCACCCC
ACCCGCCGACATCTGGGACGACTACATCTCCCGTCCCGCTCACT
ACCAAGGCCTGCTAGCCACGCTGCTCGGCCGCGACATCGAAGGC
GCAGACGAACTCCTCGACGCCACCACCCTCCAAAAAATCAGAGA
CCTCACCCTCGACAAAACTCA TCTCACCGACCTCCACCTCC G
CGGATACCAATCATTCGGCGCCCGCTTCG CCATCATCCAAAAG
AAAACCCTCCTCGGCGACGACATGGGACTCGGCAAAACAGTCCA
AGCCCTCTCCGCAGCTGCACACCTTGCCGCCACCGAAAAAGACT
TCCGCACCCTCGTCGTCGTACCCGCATCCGTCATTGTTAACTGG
ACCCGCGAATGCAAACGCTTCCTCAACCTCCCCGTATTCATCGC
CCACGGAGACAACAAACAAGACGCCATCAACGCCTGGTCTAACA
CCAACGGAATCGCAATCTGCACCTACGACGGCGTCCGCACCATG
GACATCCCCGCGCCGGGTCTGGTCATTGCCGATGAAGCCCACCT
GATCAAAAACCCCTCCACCAAACGCACCCAAGCACTGCGCAAAC
TTATCGACGCCGCCCCATACACCCTTCTGATGACCGGCACACCA
CTAGAAAACAAAGTGGAAGAGTTTGTAAATCTCGTGCGCTACAT
CCAACCGGAGCTGATCACCCGTGGCATGTCCAAAATGCAGGCCG
AGAATTTCCGCGAGCGCATCGCACCAGCCTATCTGCGCAGAAAT
CAAGCTGATGTGCTTGACGAACTCCCAGAGCGCACCGACTCCAT
CGACTGGATCGACCTCACCCCAGAAGACCGCAGCGCCTACGACG
ACCAAGTCCGCCAAGGCAGCTGGATGGGCATGCGCCGCTCCGCC
ATGCTCTCACCAACACCACGCCTAACTTCCGCAAAAATGCAACG
CATCCTAGAACTCTTCGAAGAAGCAGAAGAACACGGCCGCAAAG
CCCTCATCTTCACCTACTTCCTCGACGTCCTCGACGAACTGGAA
AAGCATCTAGGCGAGCGCGTCATCGGCCGCATTTCCGGCGACGT
GCCAGCCACCAAGCGCCAATTGCTTGTCGACGCCCTGTCCCACT
CCAAACCCGGATCCGCCCTCATTGCCCAAATCACCGCCGGGGGA
GTAGGCCTAAACATCCAATCCGCGAGCCTATGCATTATTTGTGA
ACCTCAAGTAAAGCCAACCATCGAACAGCAGGCCGTCGCCCGAG
TCCACCGCATGGGCCAAACCGCCACCGTCCAAGTCCACCGACTC
ATCGGCGACGAAACCGCAGACGAACGCATGCTAGAAATCCTGGC
AGGCAAAACTCACGTCTTCGACGTCTACGCCCGGCTATCTGAAA
CCGCAGAGATTCCAGATGCTGTGGATATCACTGAATCACAGCTG
GCAGCACGGGTTATTGATGAGGAGCGTGCACGGTTAGGGCTTAC
TGAATCCACTGGCCCTAAAGATGAAGAAACGGCC TTAAGCTAG
SEQ ID NO: 30: NCgl1575^{A1775T} ORF sequence:
ATGGCAGAATCAAACGCTATGGACCGGGCACAAATCTCTGCACT
GCTAGATAGAGCACAGCACACAATCAACCTTGCCGAACAAGCAA
ACAACGTGCTCCGACTGTTGAAAACACCCGGAACGGCCACAGTA
GGGGACAACGGGACACTCGGCACCGATACCTATCTGATCCCATC
CCGCAACATCACCTGGCCTGACAACCTGTATGTCAACGTCTTTC
TAGACGGCATGAATGCAGAAGCCACCCTTACCGATTACGTCGCA
TCAGTCGCTTCGATCCCACGCCTATGCCAGATCATCAACGAGGG
CCAAGGCGGCATGTTCCGCAGACTATTCAACCCCACCAAGGTCC
AAGCCGGCGACCAAGCTGTCTTCGACCTCATGGTCAAACTCGAC
GAGATTTCATCTACCACCCACGAAGTCTCCCGCATGCTCGAGGG
CGTCCACGCTGCCCGCACCCGCCAACAACAAGGCGTTGCACTTT
TCCCAGGTATTCATGGAGTGGGAGAGCGCTACATCGAACGCGCA
CAACAGGTACTCGCCTCAGCCCTCGGTATCGCTGGATTCGGTGC
CGAACCCTGGGACGGACATACCCTTGCCCAAGCGCGCCGGGTAG
TCCAACGCTACGCCCAAGATCCTAACTCCGAATACCGGCTGAAA
AGCGAAGCCGAGAAACACCTCACATCCATCAACGAGCTCCGCGT
ACAGATACTCCTCGAACAACTCCCCGTTGATGCCCTACGCATGG
CTACCGACCACCGCCTGCGCTTTGGATCCCTCGATTCCATCCAC
GTCGCAACCGTCGCCGACGTCCTAAAAACACACACCTCCATCCT
CACCACCGTGCAAGGTATCGGCGCCCAAACCGCGGGGCGGATGA
AAGCCGCAGCAGAAACACTCAAACAAGAAGCACTACGCCGCCAA
AACACCTCCATCGGCGACGAACCTACCCAACCCGCCATGCGTCT
AATCAACGTGCTGGCCCGCTTCGACCAAACCGAAACCATCACGC
CCGAAGAACGCGCCCGCCGCACCCGCGTCATCGACTACGTAGAA
CACATACCCCCAAGCCTCGACCCCTACATCGTCATCAACCCAGC
AACGCCTGAGTTCAACAACTTCACCGACGACCTCCGCTGGATCG
ACGCAA ACCCCAACCTCTTCCACCCACAAACAATCACCACCCC
ACCCGCCGACATCTGGGACGACTACATCTCCCGTCCCGCTCACT
ACCAAGGCCTGCTAGCCACGCTGCTCGGCCGCGACATCGAAGGC
GCAGACGAACTCCTCGACGCCACCACCCTCCAAAAAATCAGAGA
CCTCACCCTCGACAAAACTCA TCTCACCGACCTCCACCTCC G
CGGATACCAATCATTCGGCGCCCGCTTCG CCATCATCCAAAAG
AAAACCCTCCTCGGCGACGACATGGGACTCGGCAAAACAGTCCA
AGCCCTCTCCGCAGCTGCACACCTTGCCGCCACCGAAAAAGACT
TCCGCACCCTCGTCGTCGTACCCGCATCCGTCATTGTTAACTGG
ACCCGCGAATGCAAACGCTTCCTCAACCTCCCCGTATTCATCGC
CCACGGAGACAACAAACAAGACGCCATCAACGCCTGGTCTAACA
CCAACGGAATCGCAATCTGCACCTACGACGGCGTCCGCACCATG
GACATCCCCGCGCCGGGTCTGGTCATTGCCGATGAAGCCCACCT
GATCAAAAACCCCTCCACCAAACGCACCCAAGCACTGCGCAAAC
TTATCGACGCCGCCCCATTCACCCTTCTGATGACCGGCACACCA
CTAGAAAACAAAGTGGAAGAGTTTGTAAATCTCGTGCGCTACAT
CCAACCGGAGCTGATCACCCGTGGCATGTCCAAAATGCAGGCCG
AGAATTTCCGCGAGCGCATCGCACCAGCCTATCTGCGCAGAAAT
CAAGCTGATGTGCTTGACGAACTCCCAGAGCGCACCGACTCCAT
CGACTGGATCGACCTCACCCCAGAAGACCGCAGCGCCTACGACG
ACCAAGTCCGCCAAGGCAGCTGGATGGGCATGCGCCGCTCCGCC
ATGCTCTCACCAACACCACGCCTAACTTCCGCAAAAATGCAACG
CATCCTAGAACTCTTCGAAGAAGCAGAAGAACACGGCCGCAAAG
CCCTCATCTTCACCTACTTCCTCGACGTCCTCGACGAACTGGAA
AAGCATCTAGGCGAGCGCGTCATCGGCCGCATTTCCGGCGACGT
GCCAGCCACCAAGCGCCAATTGCTTGTCGACGCCCTGTCCCACT
CCAAACCCGGATCCGCCCTCATTGCCCAAATCACCGCCGGGGGA
GTAGGCCTAAACATCCAATCCGCGAGCCTATGCATTATTTGTGA
ACCTCAAGTAAAGCCAACCATCGAACAGCAGGCCGTCGCCCGAG
TCCACCGCATGGGCCAAACCGCCACCGTCCAAGTCCACCGACTC
ATCGGCGACGAAACCGCAGACGAACGCATGCTAGAAATCCTGGC
AGGCAAAACTCACGTCTTCGACGTCTACGCCCGGCTATCTGAAA
CCGCAGAGATTCCAGATGCTGTGGATATCACTGAATCACAGCTG
GCAGCACGGGTTATTGATGAGGAGCGTGCACGGTTAGGGCTTAC
TGAATCCACTGGCC CTAAAGATGA AGAAACGGCCTTAAGCTA
G
SEQ ID NO: 31: NCgl1575 wild-type coding
protein sequence:
MAESNAMDRAQISALLDRAQHTINLAEQANNVLRLLKTPGTATV
GDNGTLGTDTYLIPSRDQAVFDLMVKLDEISSTTHEVSRMLEGV
HAARTRQQQGVALFPGIHGVGERYIERAQQVLASALGIAGFGAE
PWDGHTLAQARRVVQRYAQDPNSEYRLKSEAEKHLTSINELRVQ
ILLEQLPVDALRMATDHRLRFGSLDSIHVATVADVLKTHTSILT
TVQGIGAQTAGRMKAAAETLKQEALRRQNTSIGDEPTQPAMRLI
NVLARFDQTETITPEERARRTRVIDYVEHIPPSLDPYIVINPAT
PEFNNFTDDLRWIDANPNLFHPQTITTPPADIWDDYISRPAHYQ
GLLATLLGRDIEGADELLDATTLQKIRDLTLDKTHLTDLHLRGY
QSFGARFAIIQKKTLLGDDMGLGKTVQALSAAAHLAATEKDFRT
LVVVPASVIVNWTRECKRFLNLPVFIAHGDNKQDAINAWSNTNG
IAICTYDGVRTMDIPAPGLVIADEAHLIKNPSTKRTQALRKLID
AAPYTLLMTGTPLENKVEEFVNLVRYIQPELITRGMSKMQAENF
RERIAPAYLRRNQADVLDELPERTDSIDWIDLTPEDRS AYDDQV
RQGSWMGMRRSAMLSPTPRLTSAKMQRILELFEEAEEHGRKAL
IFTYFLDVLDE LEKHLGERVIGRISGDVPATKRQLLVDALSHSK
PGSALIAQITAGGVGLNIQSASLCIICEPQVKPTIEQQAVARVH
RMGQTATVQVHRLIGDETADERMLEILAGKTHVFDVYARLSETA
EIPDAVDIT ESQLAARVID EERARLGLTE STGPKDEETA LS
SEQ ID NO: 32: NCgl1575^{Y592F} coding protein
sequence:
MAESNAMDRAQISALLDRAQHTINLAEQANNVLRLLKTPGTATV
GDNGTLGTDTYLIPSRNITWPDNLYVNVFLDGMNAEATLTDYVA
SVASIPRLCQIINEGQGGMFRRLFNPTKVQAGDQAVFDLMVKLD
EISSTTHEVSRMLEGVHAARTRQQQGVALFPGIHGVGERYIERA
QQVLASALGIAGFGAEPWDGHTLAQARRVVQRYAQDPNSEYRLK
SEAEKHLTSINELRVQILLEQLPVDALRMATDHRLRFGSLDSIH
VATVADVLKTHTSILTTVQGIGAQTAGRMKAAAETLKQEALRRQ
NTSIGDEPTQPAMRLINVLARFDQTETITPEERARRTRVIDYVE
HIPPSLDPYIVINPATPEFNNFTDDLRWIDANPNLFHPQTITTP
PADIWDDYISRPAHYQGLLATLLGRDIEGADELLDATTLQKIRD
LTLDKTHLTDLHLRGYQSFGARFAIIQKKTLLGDDMGLGKTVQA
LSAAAHLAATEKDFRTLVVVPASVIVNWTRECKRFLNLPVFIAH
GDNKQDAINAWSNTNGIAICTYDGVRTMDIPAPGLVIADEAHLI
KNPSTKRTQALRKLIDAAPFTLLMTGTPLENKVEEFVNLVRYIQ
PELITRGMSKMQAE NFRERIAPAY LRRNQADVLD ELPERTD
SIDWIDLTPEDRS AYDDQVRQGSWMGMRRSAMLSPTPRLTSAKM
QRILELFEE AEEHGRKALIFTYFLDVLDE LEKHLGERVIGRIS
GDVPATKRQLLVDALSHSKPGSALIAQITAGGVGLNIQSASLCI
ICEPQVKPTIEQQAVARVHRMGQTATVQVHRLIGDETADERMLE
ILAGKTHVFDVYARLSETAEIPDAVDIT ESQLAARVID EERA
RLGLTE STGPKDEETA LS
SEQ ID NO: 57: wild-type promotor sequence:
ttcagggtag ttgactaaag agttgctcgc gaagtagcac
ctgtcacttt tgtctcaaatattaaatcga atatcaatat
atggtctgtt tattggaacg cgtcccagtg gctgagacgc
atccgctaaa gccccaggaa ccctgtgcag aaagaaaaca
ctcctctggc taggtagacacagtttataa aggtagagtt
gagcgggtaa ctgtcagcac gtagatcgaa aggtgcacaaag
SEQ ID NO: 58: mutated promotor sequence:
ttcagggtag ttgactaaag agttgctcgc gaagtagcac
ctgtcacttt tgtctcaaatattaaatcga atatcaatat
atggtctgtt tattggaacg cgtcccagtg gctgagacg
catccgctaaa gccccaggaa ccctgtgcag aaagaaaaca
ctcctctggc taggtagacacagtttataa aggtataatt
gagcgggtaa ctgtcagcac gtagatcgaa aggtgcacaaag

### DEFINITION OF TERMS

In the present invention, the term “a bacterium with L-amino acid production capacity” refers to the ability to produce and accumulate L-amino acids of interest in culture medium and/or cells of the bacterium to the following extent, such that the bacterium producing L-amino acid can be collected when the bacterium is cultured in the culture medium. The bacterium with L-amino acid production capacity can be a bacterium that can accumulate L-amino acids of interest in culture medium and/or cells of the bacterium in an amount greater than that can be obtained by an unmodified strain.

Examples of L-amino acids include basic amino acids, such as L-lysine, L-ornithine, L-arginine, L-histidine, and L-citrulline; aliphatic amino acids, such as L-isoleucine, L-alanine, L-valine, L-leucine, and glycine; amino acids as hydroxy monoamino carboxylic acids, such as L-threonine and L-serine; cyclic amino acids, such as L-proline; aromatic amino acids, such as L-phenylalanine, L-tyrosine, and L-tryptophan; sulfur containing amino acids, such as L-cysteine, L-cystine, and L-methionine; acidic amino acids, such as L-glutamate and L-aspartate; and amino acids with amide groups in side chain, such as L-glutamine and L-asparagine. Specific examples of L-amino acids include L-glutamic acid, L-lysine, L-threonine, L-arginine, L-histidine, L-isoleucine, L-valine, L-leucine, L-phenylalanine, L-tyrosine, L-tryptophan, and L-cysteine. More specific examples of L-amino acids include L-glutamate, L-lysine, 1-threonine, and L-tryptophan. Yet more specific examples of L-amino acids include L-glutamate and L-lysine.

In the present invention, unless otherwise specified, the term “amino acid” refers to L-amino acid. In the present invention, unless otherwise specified, the term “L-amino acid” refers to L-amino acid in free form, its salt or mixture thereof.

The term “unmodified strain” refers to a control strain that has not been modified in such a way that it has specific characteristics. That is, examples of unmodified strains include wild-type strains and parental strains.

The term “homology” refers to the percentage identity between two polynucleotides or two polypeptide modules. Sequence homology between one module and another module can be determined by using methods known in the art. For example, such sequence homology can be determined by BLAST algorithm.

The term “operably linking” refers to a functional linking between a regulatory sequence and a polynucleotide sequence, whereby the regulatory sequence controls the transcription and/or translation of the polynucleotide sequence. The regulatory sequence can be a strong promoter that can improve the expression level of polynucleotides. The regulatory sequence may be a promoter derived from a microorganism belonging to Corynebacterium genus or may be a promoter derived from other microorganisms. For example, the promoter may be a trc promoter, gap promoter, tac promoter, T7 promoter, lac promoter, trp promoter, araBAD promoter, or cj7 promoter.

The term “vector” refers to a polynucleotide construct that contains the regulatory sequence of a gene and the gene sequence and is configured to express a target gene in a suitable host cell. Alternatively, a vector can also refer to a polynucleotide construct, which contains sequences that can be used for homologous recombination, so that due to the vector introduced into the host cell, the regulatory sequence of the endogenous gene in the host cell's genome can be changed, or the target gene that can be expressed can be inserted into a specific site of the host's genome. In this regard, the vector used in the present invention may further comprise a selection marker to determine the introduction of the vector into the host cell or the insertion of the vector into the chromosome of the host cell. Selection markers may include markers conferring selectable phenotypes, such as drug resistance, auxotrophic type, resistance to cytotoxic agents, or expression of surface proteins. In environments treated with such selection agents, transformed cells can be selected because cells expressing only selection markers can survive or display different phenotypic traits.

As used herein, the term “transformation” refers to the introduction of polynucleotides into host cells, so that polynucleotides can be used as extragenomic elements or inserted into the genome of host cells for replication. The method of transforming the vectors used in the present invention may include a method of introducing nucleic acids into cells. In addition, as disclosed in the related technology, an electric pulse method can be implemented based on host cells.

### Beneficial Effects

The invention finds that the product encoded by the gene has an impact on the production capacity of amino acids by weakening or knocking out the NCgl0609 gene or NCgl1575 gene. The recombinant strain is obtained by introducing point mutations into the coding sequence, or increasing the number of copies or overexpression of the gene. Compared with the wild-type strain, the obtained strain is conducive to the production of high concentrations of amino acids. Moreover, the recombinant strain was obtained by introducing point mutations into the promoter region of lysC gene. Compared with the unmutated strain, the obtained strain can also significantly improve the production of L-lysine, further to improve the generation efficiency, reduce the generation cost, and facilitate popularization and application.

### THE DETAILED EMBODIMENTS

Hereinafter, the technical solution of the present invention will be further described in detail in combination with specific examples. It should be understood that the following examples are merely illustrative and explanatory of the invention and should not be construed as limiting the scope of protection of the invention. All technologies realized based on the above contents of the present invention are fallen into the scope of the present invention. Unless otherwise stated, all raw materials and reagents used in the following examples are commercially available products or can be prepared by known methods. All operations are known in the art, or performed according to the user manual of commercially available products.

In the following examples, the basic medium used for culturing the strains have the same composition, and sucrose, kanamycin or chloramphenicol etc. are added to such basic medium composition when necessary. The basic medium composition is as follows:

| Ingredients | Formulation |
|---|---|
| Sucrose | 10 g/L |
| Polypeptone | 10 g/L |
| Beef paste | 10 g/L |
| Yeast powder | 5 g/L |
| Urea | 2 g/L |
| Sodium chloride | 2.5 g/L |
| Agar powder | 20 g/L |
| pH | 7.0 |
| Culture temperature | 32° C. |

The preparation and conditions of SSCP electrophoresis PAGE in the following examples are as follows:

| | Amount (the final concentration of |
|---|---|
| Ingredients | acrylamide is configured as 8%) |
| 40% acrylamide | 8 ml |
| ddH₂O | 26 ml |
| glycerol | 4 ml |
| 10*TBE | 2 ml |
| TEMED | 40 ul |
| 10% AP | 600 ul |
| Electrophoresis | The electrophoresis tank is placed in |
| conditions | ice and 1 × TBE buffer is used, voltage: |
| | 120 V, electrophoresis time: 10 h |

In the following examples, the fermentation medium and fermentation process of L-lysine are shown in Table 1 and 2 below:

TABLE 1
L-lysine fermentation medium formulation

| Ingredients | formulation |
|---|---|
| Starch hydrolysis sugar | 30 g/L |
| Ammonium sulfate | 12 g/L |
| Magnesium sulfate | 0.87 g/L |
| Molasses | 20 g/L |
| Acidified corn steep liquor | 3 mL/L |
| Phosphoric acid | 0.4 mL/L |
| Potassium chloride | 0.53 g/L |
| Defoamer (2% GPE) | 4 mL/L |
| Ferrous sulfate | 120 mg/L |
| Manganese sulfate | 120 mg/L |
| Nicotinamide | 42 mg/L |
| Calcium pantothenate | 6.3 mg/L |
| Vitamin B1 | 6.3 mg/L |
| Copper and zinc salt solutions | 0.6 g/L |
| Biotin | 0.88 mg/L |

TABLE 2
L-lysine fermentation control process

| Corrected | Temperature: 37° C., air velocity: 4 L/min, rotational speed: 1000 rpm, tank | | |
|---|---|---|---|
| DO100% | pressure: 0 mpa, calibrated after 5 min | | |
| Inoculation | 10% | Culture temperature ° C. | 37° C. |
| amount | | | |
| pH | pH 6.9 ± 0.05 | Dissolved oxygen DO | 10-30% |

| | |
|---|---|
| initial condition | Temperature 37° C., pH 6.9, tank pressure: 0 Mpa, air elocity: 3 L/min, rotational |
| | speed: 550 rpm |
| Full control | Full range control: 1. When the dissolved oxygen is less than 30%, the rotational |
| | speed is increased to 750 rpm → 800 rpm → air velocity 4 l/min → 850 rpm → |
| | 950 rpm in turn; 2. Fermentation for 6 h, tank pressure is increased by 0.01 Mpa; |
| | 12 h tank pressure is increased by 0.02 MPa → 0.03 MPa → 0.04 MPa → 0.05 Mpa |
| Residual sugar | 0.1-0.2% before F12 h; After F12 h, in combination with DO, it is required to |
| control | control residual sugar as 0.1-0.05% |
| Ammonia | 0.1-0.15 before F12 h; 0.15-0.25 in F12-F32 h; 0.1-0.15 after F32 h |
| nitrogen | |
| control | |
| Feed material | 25% ammonia, 70% concentrated sugar, 50% ammonium sulfate, 10% PGE |
| Fermentation | About 48 h |
| cycle | |

In the following examples, the fermentation medium and fermentation process of L-glutamate are shown in Table 3 and 4 below:

TABLE 3
L-glutamate fermentation medium formulation

| Reagent names | proportioning |
|---|---|
| glucose | 5.0 g/L |
| phosphoric acid | 0.38 g/L |
| Magnesium | 1.85 g/L |
| sulfate | |
| potassium | 1.6 g/L |
| chloride | |
| Biotin | 550 μg/L |
| Vitamin B1 | 300 μg/L |
| ferrous sulfate | 10 mg/L |
| Manganese | 10 g/dl |
| sulfate | |
| KH₂PO₄ | 2.8 g/L |
| Vitamin C | 0.75 mg/L |
| Vitamin B12 | 2.5 μg/L |
| P-aminobenzoic | 0.75 mg/L |
| acid | |
| Defoamer | 0.0015 ml/dl |
| betaine | 1.5 g/L |
| Cane Molasses | 7 ml/L |
| Corn steep | 77 ml/L |
| liquor | |
| Aspartic acid | 1.7 g/L |
| Hair powder | 2 g/L |

TABLE 4
L-glutamate fermentation control process

| | conditions | | | |
|---|---|---|---|---|
| | | | | Culture |
| Periods | Revolutions | Air velocity | pressure | temperature |
| 0 h | 400 rpm | 3 L/min | 0.05 MPA | 32.5° C. |
| OD 1.0 | 600 rpm | 5 L/min | 0.08 MPA | 37° C. |
| OD 1.4 | 700 rpm | 7 L/min | 0.11 MPA | 38° C. |

| | |
|---|---|
| 32 h~34 h | At the end of fermentation, 50~20% dissolved oxygen is used as the |
| | standard for increasing and decreasing air velocity in the control |
| | process |
| PH | 0 h control: pH 7.0, 14 h control: pH 6.8 |
| Feed sugar | The concentration of feed sugar in the fermentation tank is 50~55%, |
| control | and the residual sugar in the fermentation tank is controlled to be |
| | 0.5~1.0% |

### Example 1: Construction of the Transformed Vector Pk18-NCgl0609^{R334*} Containing the Coding Region of NCgl0609 Gene with Point Mutation

Based on the genome sequence of Corynebacterium glutamicum ATCC13032 published by NCBI, two pairs of primers for amplifying the coding region sequence of NCgl0609 gene were designed and synthesized. Point mutation was introduced into the coding region of NCgl0609 gene (SEQ ID NO: 1, and the corresponding amino acid sequence encoding the proteins is SEQ ID NO: 3) in the background of strain YP97158 [Depositary No.: CGMCC No.12856, Depositary date: Aug. 16, 2016, Depositary unit: Institute of Microbiology, Chinese Academy of Sciences, No. 3, Yard. 1, Beichen West Road, Chaoyang District, Beijing, Tel: 010-64807355, recorded in Chinese patent application CN106367432A (the filing date: Sep. 1, 2016, and the publication date: Feb. 1, 2017), and it is confirmed via sequencing that the wild type NCgl0609 gene was retained in the chromosome of the strain] by means of allelic replacement, and thus the nucleotide sequence of NCgl0609 gene at position 1000 was changed from C to T (SEQ ID NO: 2), and the corresponding amino acid sequence encoding proteins at position 334 was changed from arginine to a terminator (SEQ ID NO: 4: NCgl0609^{R334*}). The primers were designed as follows (synthesized by Shanghai Invitrogen Company):

P1:
(SEQ ID NO: 5)
5′ CAGTGCCAAGCTTGCATGCCTGCAGGTCGACTCTAG
GGACGGCAACGTACATAAC3′;
P2:
(SEQ ID NO: 6)
5′ GTTGCCGGTGAGTCAAACAGTCATTTTGC 3′;
P3:
(SEQ ID NO: 7)
5′ GCAAAATGACTGTTTGACTCACCGGCAAC 3′;
and
P4:
(SEQ ID NO: 8)
5′ CAGCTATGACCATGATTACGAATTCGAGCTCGGTACCC
GCGGCTG GAAATGTGGAG3′.

Construction method: Corynebacterium glutamicum ATCC13032 was used as the template, and primers P1 and P2, P3 and P4 were used, respectively, for PCR amplification.

PCR System: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., (denaturation for 30 s at 94° C., annealing for 30 s at 52° C., extension for 40 s at 72° C., 30 cycles), and over extension for 10 min at 72° C., and then two DNA fragments containing the coding region of NCgl0609 gene in sizes of 698 bp and 648 bp respectively (NCgl0609 Up and NCgl0609 Down) were obtained. After the two DNA fragments were separated and purified via agarose gel electrophoresis, the two DNA fragments as templates were amplified into 1317 bp fragments by overlap PCR with P1 and P4 as primers.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., (denaturation for 30 s at 94° C., annealing for 30 s at 52° C., extension for 60 s at 72° C., 30 cycles), and over extension for 10 min at 72° C. This DNA fragment resulted in the change of cytosine (C) at position 1000 in the coding region of YP97158 NCgl0609 gene into thymine (T), and finally resulted in the 334th amino acid encoding the protein changed from arginine (R) to a terminator. This DNA fragment was purified via agarose gel electrophoresis, and was linked with the pK18mobsacB plasmid (purchased from Addgene company, double enzyme digested with Xbal I/BamH I, respectively) which was double enzyme digested and then purified with NEBuilder enzyme (purchased from NEB company) at 50° C. for 30 min, and a positive vector pk18-NCgl0609^{R334*} was obtained from the monoclone grown after the transformation of the linked product by pcr identification, and this plasmid contained a kanamycin resistance marker. The vector pk18-NCgl0609^{R334*} with correct enzyme digestion was sent to the sequencing company for sequencing and identification, and the vector pk18-NCgl0609^{R334*} containing correct point mutation (C-T) was stored for use.

### Example 2: Construction of an Engineered Strains of NCgl0609^{R334*} Containing Point Mutation

Construction method: the allelic replacement plasmid pk18-NCgl0609^{R334*} was transformed into L-lysine production strain YP97158 by electric shock (See WO2014121669A1 for its construction method; it is confirmed by sequencing that the coding region of wild type NCgl0609 gene is reserved in the chromosome of the strain). The single colony produced by culturing was identified by primer P1 and universal primer M13R, and the strain that can amplify bands in size of 1375 bp was a positive strain. The positive strain was cultured on the medium containing 15% sucrose, the single colony produced by culturing was cultured on the medium containing kanamycin and the medium without kanamycin, respectively, and the strains that grew on the medium without kanamycin but did not grow on the medium containing kanamycin were further identified by PCR with the following primers (synthesized by Shanghai Invitrogen Company):

P5:
(SEQ ID NO: 9)
5′ CTAGCCGGTTCCAGTCAG 3′;
and
P6:
(SEQ ID NO: 10)
5′ GGACGTCTGTTCACCATTG 3′.

The above PCR amplification product was 264 bp, which was denatured at 95° C. for 10 min and subjected to ice bath for 5 min followed by sscp electrophoresis (the plasmid pk18-NCgl0609^{R334*} amplification fragment was used as the positive control, YP97158 amplification fragment was used as the negative control, and the water was used as the blank control). Due to different fragment structures and electrophoresis positions, the strains whose electrophoresis positions are different from those of negative control fragments and are consistent with those of positive control fragments are the strains with successful allelic replacement. The NCgl0609 fragment of the positive strain was subjected to PCR amplification using primer P5/P6, and was linked to PMD19-T vector for sequencing. Through sequence alignment, the strain with mutation (C-T) of base sequence was the positive strain with successful allelic replacement, and was named as YPL-4-041.

### Example 3: Construction of Engineering Strains Overexpressing NCgl0609 and NCgl0609^{R334*} Genes in Genome

Based on the genome sequence of wild type Corynebacterium glutamicum ATCC13032 published by NCBI, three pairs of primers for amplifying the upstream and downstream homologous arm fragments and the coding region and promoter region sequences of NCgl0609 and NCgl0609^{R334*} gene were designed and synthesized, and NCgl0609 or NCgl0609^{R334*} gene was introduced into strain YP97158 by way of homologous recombination.

Primers were designed as follows (synthesized by Shanghai Invitrogen Company):

P7:
(SEQ ID NO: 11)
5′ CAGTGCCAAGCTTGCATGCCTGCAGGTCGACTCTAG
AATGCGTTCTG GACTGAGG 3′;
P8:
(SEQ ID NO: 12)
5′ CAGTGCCAAGCTTGCATGCCTGCAGGTCGACTCTAG
AATGCGTTCTG GACTGAGG 3′;
P9:
(SEQ ID NO: 13)
5′ CATCTGTTCTCGGTGCACCAGCTGCGAGGATCATC
TC 3′;
P10:
(SEQ ID NO: 14)
5′ GATTTAATTGCGCCATCTGATTCTGGCAACAACTC
CTTCCTTGACC 3′;
P11:
(SEQ ID NO: 15)
5′ GGTCAAGGAAGGAGTTGTTGCCAGAATCAGATGGC
GCAATTA AATC AAG 3′;
and
P12:
(SEQ ID NO: 16)
5′ CAGCTATGACCATGATTACGAATTCGAGCTCGGT
ACCCGCTATGACACCTTCAACGGATC 3′.

Construction method: Corynebacterium glutamicum ATCC13032 or YPL-4-041 was used as template, respectively, for PCR amplification with primers P7/P8, P9/P10, P11/P12, to obtain the upstream homologous arm fragment of 768 bp, NCgl0609 or NCgl0609^{R334*} gene and its promoter fragment of 1626 bp and the downstream homologous arm fragment of 623 bp. After the completion of PCR reaction, the three amplified fragments were electrophoretically recovered using a column DNA gel recovery kit, respectively. The recovered three fragments were linked with the pK18mobsacB plasmid (purchased from Addgene Company, double enzyme digested with Xbal I/BamH I, respectively) which was double enzyme digested and then purified with NEBuilder enzyme (purchased from NEB Company) at 50° C. for 30 minutes, and a positive integrated plasmid was obtained from the monoclone grown after the transformation of the linked product by pcr identification. This plasmid contained a kanamycin resistance marker, and the recombinant with plasmid integrated into the genome can be obtained through kanamycin screening.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., denaturation for 30 s at 94° C., annealing for 30 s at 52° C., extension for 60 s at 72° C. (30 cycles), and over extension for 10 min at 72° C. The correctly sequenced integrated plasmid was electrotransformed into the L-lysine production strain YP97158. The single colony produced by culturing was identified by PCR with primers P13/P14. The strain that amplified fragment with 1317 bp by PCR was a positive strain, and the strain without fragment by amplified was original strain. The positive strain was cultured on the medium containing 15% sucrose, and the single colony produced by culturing was further identified by PCR with primers P15/P16. The bacteria amplifying fragment of 1352 bp were positive strains with NCgl0609 or NCgl0609^{R334*} gene integrated into the YP97158 genome, which were named YPL-4-042 (without mutation site) and YPL-4-043 (with mutation site).

P13:
(SEQ ID NO: 17)
5′ TCCAAGGAAGATACACGCC 3′;
P14:
(SEQ ID NO: 18)
5′ CGAAATGGAAGTTGTGCG 3′;
P15:
(SEQ ID NO: 19)
5′ CGATGATGCCGATTACCTC 3′;
P16:
(SEQ ID NO: 20)
5′ CGTTGGAATCTTGCGTTG 3′.

### Example 4: Construction of Engineering Strains Overexpressing NCgl0609 or NCgl0609^{R334*} Genes in Plasmid

Based on the genome sequence of wild type Corynebacterium glutamicum ATCC13032 published by NCBI, a pair of primers for amplifying the coding region and promoter region sequences of NCgl0609 or NCgl0609^{R334*} gene were designed and synthesized. The primers were designed as follows (synthesized by Shanghai Invitrogen Company):

P17:
(SEQ ID NO: 21)
5′GCTTGCATGCCTGCAGGTCGACTCTAGAGGATCCCC
CAGCTGCGAGG A TCATCTC 3′;
and
P18:
(SEQ ID NO: 22)
5′ATCAGGCTGAAAATCTTCTCTCATCCGCCAAAAC
CAACAACTCCTTCCTTGACC3′.

Construction method: Corynebacterium glutamicum ATCC13032 and YPL-4-041 were used as template, respectively, for PCR amplification with primers P17/P18 to obtain NCgl0609 and NCgl0609^{R334*} genes and their promoter fragments of 1582 bp. The amplified products were subjected to electrophoresis and purified using a column DNA gel recovery kit. The recovered DNA fragment and a shuttle plasmid pXMJ19 recovered by EcoR I enzyme digestion were linked at 50° C. with NEBuilder enzyme (purchased from NEB) for 30 min, and the positive overexpression plasmids pXMJ19-NCgl0609 and pXMJ19-NCgl0609^{R334*} were obtained from the monoclones grown after the transformation of the linker products by pcr identification with primer M13, and then these plasmids were sent to sequencing. Because the plasmid contained a chloramphenicol resistance marker, chloramphenicol can be used to screen whether the plasmid was transformed into the strain.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL. The PCR amplification was carried out as follows: pre-denaturation for 5 min at 94° C., denaturation for 30 s at 94° C., annealing for 30 s at 52° C., extension for 60 s at 72° C. (30 cycles), and over extension for 10 min at 72° C.

The correctly sequenced pXMJ19-NCgl0609 and pXMJ19-NCgl0609^{R334*} plasmids were electrotransformed into the L-lysine production strain YP97158, respectively. The single colony produced by culturing was identified by PCR with primers M13F/P18. The strains amplifying fragment with 1585 bp by PCR were positive strains, which was named YPL-4-044 (without mutation site) and YPL-4-045 (with mutation site).

### Example 5: Construction of Engineered Strains with NCgl0609 Gene Deleted in Genome

Based on the genome sequence of Corynebacterium glutamicum ATCC13032 published by NCBI, two pairs of primers for amplifying the fragments at two ends of the coding region of NCgl0609 gene were designed and synthesized, as upstream and downstream homologous arm fragments The primers were designed as follows (synthesized by Shanghai Invitrogen Company):

P19:
(SEQ ID NO: 23)
5′ CAGTGCCAAGCTTGCATGCCTGCAGGTCGACTCTAG
AATGAA TGG GATGGGTCG 3′;
and
P20:
(SEQ ID NO: 24)
5′ CATCATCGGTTACTCTGGCCGAAATGGAAGTTGTGCG 3′;
P21:
(SEQ ID NO: 25)
5′ CGCACAACTTCCATTTCGGCCAGAGTAACCGATGATG 3′;
P22:
(SEQ ID NO: 26)
5′CAGCTATGACCATGATTACGAATTCGAGCTCGGTACCC
TCAACAACTCCTCCTTGACC3′.

Construction method: Corynebacterium glutamicum ATCC13032 was used as template for PCR amplification with primers P19/P20 and P21/P22, respectively, to obtain upstream homologous arm fragment of 661 bp and downstream homologous arm fragment of 692 bp. Then primers P19/P22 were used for OVERLAP PCR to obtain the whole homologous arm fragment of 1334 bp. The amplified products were subjected to electrophoresis and purified using a column DNA gel recovery kit. The recovered DNA fragments were linked with the pK18mobsacB plasmid (purchased from Addgene Company, double enzyme digested with Xbal I/BamH I, respectively) which were double enzyme digested and then purified with NEBuilder enzyme (purchased from NEB Company) at 50° C. for 30 minutes. Positive knockout vector pK18-ΔNCgl0609 were obtained from the monoclones grown after the transformation of the linker products by pcr identification with primer M13, and then these plasmids were sent to sequencing. The plasmid contained kanamycin resistance as a screening marker.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL. The PCR amplification was carried out as follows: pre-denaturation for 5 min at 94° C., denaturation for 30 s at 94° C., annealing for 30 s at 52° C., extension for 90 s at 72° C. (30 cycles), and over extension for 10 min at 72° C.

The correctly sequenced knockout plasmid pK18-ΔNCgl0609 was electrotransformed into lysine producing strain YP97158, and the single colony produced by culturing was identified by PCR with the following primers (synthesized by Shanghai Invitrogen Company):

P23:
(SEQ ID NO: 27)
5′ AATGAATGG GATGGGTCG 3′;
and
P24:
(SEQ ID NO: 28)
5′ CAACAACT CCT TCCTTGACC 3′.

The strains simultaneously amplifying 1334 bp and 1788 bp bands by the above PCR were positive strains, and the strains only amplifying 1788 bp band were original strains. After screening on the 15% sucrose medium, the positive strains were cultured on the medium containing kanamycin and the medium without kanamycin, respectively. The strains that grew on the medium without kanamycin but did not grow on the medium containing kanamycin were further identified by PCR using primers P23/P24. The strains amplifying 1334 bp band were the positive strains whose NCgl0609 gene coding region was knocked out. Again, the positive strain NCgl0609 fragment was PCR amplified with primers P23/P24 and linked to PMD19-T vector for sequencing. The correctly sequenced strain was named YPL-4-046.

### Example 6: L-Lysine Fermentation Experiment

The strains constructed from Examples 2-5 and the original strain YP97158 were performed a fermentation experiment in the BLBIO-5GC-4-H fermentation tank (purchased from Shanghai Bailun Biotechnology Co., Ltd.) with the culture medium shown in Table 1 and the control process shown in Table 2. Each strain was repeated three times, and the results are shown in Table 5.

TABLE 5
Results of L-lysine fermentation experiment

| Strains | L-Lysine production (g/100 ml) | OD(660 nm) |
|---|---|---|
| YP97158 | 18.9 | 37.3 |
| YPL-4-041 | 19.3 | 38.1 |
| YPL-4-042 | 19.2 | 37.8 |
| YPL-4-043 | 19.5 | 38.4 |
| YPL-4-044 | 19.4 | 37.7 |
| YPL-4-045 | 19.7 | 38.3 |
| YPL-4-046 | 18.0 | 36.8 |

The results are as shown in Table 5. Point mutation NCgl0609^{R334*} and overexpression of NCgl0609 gene coding region in Corynebacterium glutamicum contribute to the increase of L-lysine production and growth rate, while weakening or knocking out the gene is not conducive to the accumulation of L-lysine, and will reduce the growth rate of the strain.

### Example 7: Introduction of NCgl0609 Gene Overexpression in Glutamate Production Strain, or Point Mutation NCgl0609^{R334*} and Overexpression in the Coding Region of NCgl0609 Gene, and Preformation of Fermentation Experiments

According to the methods of Examples 1-5, using the same primers and experimental conditions, Corynebacterium ATCC13869 was used as the starting bacterium, and the bacterium of ATCC 13869 was used as expression bacterium to obtain the glutamate production engineering strains YPG-013 containing point mutated NCgl0609^{R334*}, the glutamate production engineering strains YPG-014 and YPG-015 overexpressing NCgl0609 and NCgl0609^{R334*} genes in the genome, the glutamate production engineering strains YPG-016 and YPG-017 overexpressing NCgl0609 and NCgl0609^{R334*} genes in the plasmid, and the glutamate production engineering strain YPG-018 that lacks NCgl0609 gene in the genome.

The strains constructed in Examples and the original strain were performed a fermentation experiment (with bacterium of ATCC 13869 as expression bacterium) in the BLBIO-5GC-4-H fermentation tank (purchased from Shanghai Bailun Biotechnology Co., Ltd.) with the culture medium shown in Table 3 and the control process shown in Table 4. Each strain was repeated three times, and the results are shown in Table 6.

TABLE 6
Results of L-glutamate fermentation experiment

| Strains | L-glutamate production (g/l) | OD(660 nm) |
|---|---|---|
| ATCC13869 | 101.0 | 42.3 |
| YPG-013 | 103.5 | 43.4 |
| YPG-014 | 103.9 | 42.8 |
| YPG-015 | 103.2 | 43.7 |
| YPG-016 | 103.6 | 42.6 |
| YPG-017 | 103.8 | 43.6 |
| YPG-018 | 98.5 | 40.5 |

The results are as shown in Table 6. Point mutation NCgl0609^{R334*} and overexpression of NCgl0609 gene coding region in Corynebacterium glutamicum contribute to the increase of L-glutamate production and growth rate, while weakening or knocking out the gene is not conducive to the accumulation of L-glutamic acid, and will reduce the growth rate of the strain.

### Example 8: Construction of Transformation Vector pK18-NCgl1575^{A1775T} Containing the Coding Region of NCgl1575 Gene with Point Mutation

Based on the genome sequence of wild type Corynebacterium glutamicum ATCC13032 published by NCBI, two pairs of primers for amplifying the coding region sequence of NCgl1575 gene were designed and synthesized. Point mutation was introduced into the coding region of NCgl1575 gene (SEQ ID NO:29) in the background of strain YP97158 (it was confirmed by sequencing that wild type NCgl1575 gene was retained in the chromosome of the strain) by means of allelic replacement. The corresponding amino acid sequence encoding the proteins was SEQ ID NO: 31, and the nucleotide sequence of NCgl1575 gene at position 1775 was changed from A to T (SEQ ID NO:30: NCgl1575^{A1775T}), and in the corresponding amino acid sequence encoding the proteins at position 592 was changed from tyrosine to phenylalanine (SEQ ID NO:32: NCgl1575 Y592F).

Primers were designed as follows (synthesized by Shanghai Invitrogen Company):

P1′:
(SEQ ID NO: 33)
5′CAGTGCCAAGCTTGCATGCCTGCAGGTCGACTCTAG
TGCGTTCGTCTGCGGTTTCG 3′;
P2′:
(SEQ ID NO: 34)
5′ ATCGACGCCGCCCCATTCACCCTTCTGATG 3′;
P3′:
(SEQ ID NO: 35)
5′ CATCAGAAGGGTGAATGGGGCGGCGTCGAT 3′;
and
P4′:
(SEQ ID NO: 36)
5′CAGCTATGACCATGATTACGAATTCGAGCTCGGTACCC
AAGCCTCGACCCCTACATC 3′.

Construction method: Corynebacterium glutamicum ATCC13032 was used as template for PCR amplification with primers P1′ and P2′, P3′ and P4′, respectively.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., denaturation for 30 s at 94° C., annealing for 30 s at 52° C., extension for 40 s at 72° C. (30 cycles), and over extension for 10 min at 72° C. Two DNA fragments containing NCgl1575 geng coding region in sizes of 766 bp and 759 bp, respectively, were obtained (NCgl1575 Up and NCgl1575 Down).

After separation and purification of the above two DNA fragments by agarose gel electrophoresis, the above two DNA fragments were used as templates, and P1′ and P4′ were used as primers, to amplify a fragment in length of about 1495 bp by overlap PCR.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., denaturation for 30 s at 94° C., annealing for 30 s at 52° C., extension for 90 s at 72° C. (30 cycles), and over extension for 10 min at 72° C.

This DNA fragment (NCgl1575^{A1775T}) resulted in the change of adenine (A) at position 1775 in the coding region of YP97158 NCgl1575 gene into thymine (T), and finally resulted in the change of amino acid at position 592 of the coding protein from tyrosine (Y) to phenylalanine (F).

The NCgl1575^{A1775T} separated and purified by agarose gel electrophoresis and the pK18mobsacB plasmid (purchased from Addgene) recovered by Xba I enzyme digestion were assembled with the NEBuider recombination system to obtain vector pK18-NCgl1575^{A1775T}, and the plasmid contained a kanamycin resistance marker. The vector pK18-NCgl1575^{A1775T} was sent to the sequencing company for sequencing and identification, and the vector pK18-NCgl1575^{A1775T} containing the correct point mutation (A-T) was stored for use.

### Example 9: Construction of Engineering Strains Containing NCgl1575^{A1775T} with Point Mutation

Construction method: The allelic replacement plasmid pK18-NCgl1575^{A1775T} was transformed into L-lysine production strain YP97158 by electric shock. The single colony produced by culturing was identified by primer P1′ and universal primer M13R, respectively. The strain that can amplify 1502 bp band was a positive strain. The positive strains were cultured on the medium containing 15% sucrose, and the single colony produced by culturing was cultured on the medium containing kanamycin and the medium without kanamycin, respectively. The strains that grew on the medium without kanamycin, but did not grow on the medium containing kanamycin were further identified by PCR with the following primers (synthesized by Shanghai Invitrogen Company):

P5′:
(SEQ ID NO: 37)
5′ CACATC AGCTTGATTT CTGC 3′;
and
P6′:
(SEQ ID NO: 38)
5′ GGTCATTGCC GATGAAGCCC 3′.

The above PCR amplification product was 256 bp, which was denatured at high temperature and subjected to ice bath, followed by sscp electrophoresis (the plasmid pK18-NCgl1575^{A1775T} amplification fragment was used as the positive control, YP97158 amplification fragment was used as the negative control, and the water was used as the blank control). Due to different fragment structures and electrophoresis positions, the strains whose electrophoresis positions are different from those of negative control fragments and are consistent with those of positive control fragments are the strains with successful allelic replacement. The fragment of interest of the strains with successful allelic replacement was subjected to PCR amplification using primer P5′ and P6′ again, and was linked to PMD19-T vector for sequencing. Through sequence alignment, the sequence in which base sequence is mutated verifies that the allelic replacement of the strain is successful, and it is named YPL-4-023.

### Example 10: Construction of Engineering Strains Overexpressing NCgl1575 or NCgl1575^{A1775T} Gene in Genome

Based on the genome sequence of wild type Corynebacterium glutamicum ATCC13032 published by NCBI, three pairs of primers for amplifying the upstream and downstream homologous arm fragments and the sequences of NCgl1575 or NCgl1575^{A1775T} gene coding region and promoter region were designed and synthesized, and NCgl1575 or NCgl1575^{A1775T} gene was introduced into strain YP97158 by homologous recombination.

Primer was designed as follows (synthesized by Shanghai Invitrogen Company):

P7′:
(SEQ ID NO: 39)
5′ CAGTGCCAAGCTTGCATGCCTGCAGGTCGACTCTAGAATG
CGTTCTGGACTGAGG 3′;
P8′:
(SEQ ID NO: 40)
5′ GAAACGGCCTTAAGCTAGGTGCACCGAG AACAGATG 3′;
P9′:
(SEQ ID NO: 41)
5′ CATCTGTTCTCGGTGCAC CTAGCTTAAG GCCGTTTC 3′;
P10′:
(SEQ ID NO: 42)
5′ CTTGATTTAATTGCGCCATCAAGCTTTTCC CGCCCGGTT 3′;
P11′:
(SEQ ID NO: 43)
5′ AACCGGGCGG GAAAAGCTTGATGGCGCAATTAAATCAAG 3′;
and
P12′:
(SEQ ID NO: 44)
5′CAGCTATGACCATGATTACGAATTCGAGCTCGGTACCC GCTAT
GACACCTTCAACGGATC 3′.

Construction method: Corynebacterium glutamicum ATCC13032 or YPL-4-023 was used as templates, respectively, for PCR amplification with primers P7′/P8′, P9′/P10′, P11′/P12′, to obtain upstream homologous arm fragment of 802 bp, NCgl1575 gene and its promoter fragment of 2737 bp, or NCgl1575^{A1775T} gene and its promoter fragment of 2737 bp, and downstream homologous arm fragment of 647 bp. Then, the above three amplified fragments (upstream homologous arm fragment, NCgl1575 gene and its promoter fragment, and downstream homologous arm fragment; or upstream homologous arm fragment, NCgl1575^{A1775T} gene and its promoter fragment, and downstream homologous arm fragment) were mixed as template for amplification with primers P7′/P12′ to obtain integrated homologous arm fragment of 4111 bp.

After the completion of PCR reaction, the amplified product is electrophoretically recovered, and the 4111 bp DNA fragment required was recovered with a column DNA gel recovery kit (TIANGEN), and was linked with the shuttle plasmid PK18mobsacB recovered by Xba I enzyme digestion using NEBBuider recombination system, to obtain the integrated plasmid PK18mobsacB-NCgl1575 or PK18mobsacB-NCgl1575^{A1775T} The plasmid contained a kanamycin resistance marker, and the recombinant with plasmid integrated into the genome can be obtained through kanamycin screening.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL. The PCR amplification was carried out as follows: pre-denaturation for 5 min at 94° C., denaturation for 30 s at 94° C., annealing for 30 s at 52° C., extension for 180 s at 72° C. (30 cycles), and over extension for 10 min at 72° C.

The two integrated plasmids were electrotransformed into the L-lysine production strain YP97158, respectively, and the single colony produced by culturing was identified by PCR with primers P13′/P14′. The strain amplifying fragments in size of 1778 bp by PCR was a positive strain, and the strain without fragments amplified was an original strain. The positive strains were screened on 15% sucrose medium and then cultured on the medium containing kanamycin and the medium without kanamycin, respectively. The strains that grew on the medium without kanamycin, but did not grow on the medium containing kanamycin were further identified by PCR with primers P15′/P16′. The bacteria amplifying fragment in size of 1756 bp were strains with NCgl1575 or NCgl1575^{A1775T} gene integrated into the YP97158 genome, which were named YPL-4-024 (without mutation site) and YPL-4-025 (with mutation site), respectively.

P13′:
(SEQ ID NO: 45)
5′ TCCAAGGAAGATACACGCC 3′;
P14′:
(SEQ ID NO: 46)
5′ CTTCTGATGA CCGGCACACC 3′;
P15′:
(SEQ ID NO: 47)
5′ TAGTCGATGA CGCGGGTGCG 3′;
and
P16′:
(SEQ ID NO: 48)
5′ CGTTGGAATCTTGCGTTG 3′.

### Example 11: Construction of Engineering Strains Overexpressing NCgl1575 or NCgl1575^{A1775T} Gene on Plasmid

Based on the genome sequence of wild type Corynebacterium glutamicum ATCC13032 published by NCBI, a pair of primers for amplifying the coding region and promoter region sequences of NCgl1575 or NCgl1575^{A1775T} gene were designed and synthesized. The primers were design as follows (synthesized by Shanghai Invitrogen Company):

P17′:
(SEQ ID NO: 49)
5′GCTTGCATGCCTGCAGGTCGACTCTAGAGGATCCCC CTAGCT
TAAG GCCGTTTC 3′;
and
P18′:
(SEQ ID NO: 50)
5′ATCAGGCTGAAAATCTTCTCTCATCCGCCAAAAC AAGCTTT
TCC CGCCCGGTT 3′

Construction method: ATCC13032 and YPL-4-023 were used as templates, respectively, for PCR amplification with primers P17′/P18′, to obtain NCgl1575 or NCgl1575^{A1775T} geng and their promotor fragments of 2749 bp. The amplified products were recovered by electrophoresis. The desired 2749 bp DNA fragments were recovered by a column DNA gel recovery kit, and were linked with the shuttle plasmid pXMJ19 recovered by EcoR I enzyme digestion using the NEBuider recombination system to obtain the overexpression plasmids pXMJ19-NCgl1575 and pXMJ19-NCgl1575^{A1775T} Plasmids containing chloramphenicol resistance markers can be obtained through chloramphenicol screening and transformed into strains.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., denaturation for 30 s at 94° C., annealing for 30 s at 52° C., extension for 120 s at 72° C. (30 cycles), and over extension for 10 min at 72° C.

The plasmids pXMJ19-NCgl1575 and pXMJ19-NCgl1575^{A1775T} were electrotransformed into the L-lysine production strain YP97158, respectively. The single colony produced by culturing was identified by PCR with primers M13 (−48) and P18′. The single colony amplifying fragment in size of 2752 bp by PCR was transformed strains which were named YPL-4-026 (without mutation site) and YPL-4-027 (with mutation site).

### Example 12: Construction of Engineering Strains with NCgl1575 Gene Deleted in Genome

Based on the genome sequence of Corynebacterium glutamicum ATCC13032 published by NCBI, two pairs of primers for amplifying fragments at two ends of the coding region of NCgl1575 gene were synthesized as upstream and downstream homologous arm fragments. Primers were design as follows (synthesized by Shanghai Invitrogen Company):

P19′:
(SEQ ID NO: 51)
5′CAGTGCCAAGCTTGCATGCCTGCAGGTCGACTCTAGACCG
GCGCAG ATGCCAACGC 3′;
P20′:
(SEQ ID NO: 52)
CCCAGAACTGAAGGTCTAATTGCCTAAGG CCGGAATT 3′;
P21′:
(SEQ ID NO: 53)
AATTCCGGCCTTAGGCAATTAGACCTTC AGTTCTGGG 3′;
and
P22′:
(SEQ ID NO: 54)
5′ CAGCTATGACCATGATTACGAATTCGAGCTCGGTACCC GCT
TGATGAA GGCTCCAG 3′.

Corynebacterium glutamicum ATCC13032 was used as a template for PCR amplification with primers P19′/P20′ and P21′/P22′, respectively, so as to obtain upstream homologous arm fragment of 775 bp and downstream homologous arm fragments of 807 bp. Then, they were subjected to overlap PCR with primers P19′/P22′ to obtain a whole homologous arm fragment of 1545 bp. After the completion of PCR reaction, the amplified product was electrophoretically recovered, and the desired 1545 bp DNA fragment was recovered using a column DNA gel recovery kit, and was linked with shuttle plasmid pk18mobsacB recovered by Xba I enzyme digestion through the NEbuider recombination system to obtain knockout plasmid. The plasmid contained a kanamycin resistant marker.

The knockout plasmid was electrotransformed into a lysine producing strain YP97158, and the single colony produced by culturing was identified by PCR with the following primers (synthesized by Shanghai Invitrogen Company):

P23′:
(SEQ ID NO: 55)
5′ ACCGGCGCAG ATGCCAACGC 3′;
and
P24′:
(SEQ ID NO: 56)
5′ GCTTGATGAA GGCTCCAG 3′.

The strains amplifying bands in size of 1471 bp and 4150 bp by above PCR were positive strains, and the strains only amplifying a band in size of 4150 were original bacteria. After screening on 15% sucrose medium, the positive strains were cultured on the medium containing kanamycin and the medium without kanamycin, respectively, and the strains that grew on the medium without kanamycin but did not grow on the medium containing kanamycin were further identified by PCR using primers P23′/P24′. The strain amplifying a band in size of 1471 bp was the engineering strain with the coding sequence of NCgl1575 gene deleted, which was named YPL-4-028.

### Example 13: L-Lysine Fermentation Experiment

The strains constructed from Examples 9-12 and the original strain YP97158 were performed a fermentation experiment in the BLBIO-5GC-4-H fermentation tank (purchased from Shanghai Bailun Biotechnology Co., Ltd.) with the culture medium shown in Table 1 and the control process shown in Table 2. Each strain was repeated three times, and the results are shown in Table 7.

TABLE 7
Results of L-Lysine fermentation experiment

| Strains | L-lysine production (g/100 ml) | OD(660 nm) |
|---|---|---|
| YP97158 | 18.8 | 37.3 |
| YPL-4-023 | 19.6 | 36.8 |
| YPL-4-024 | 19.6 | 37.0 |
| YPL-4-025 | 19.8 | 35.5 |
| YPL-4-026 | 19.3 | 36.3 |
| YPL-4-027 | 19.7 | 37.2 |
| YPL-4-028 | 18.0 | 36.8 |

The results are as shown in Table 7. Overexpression of NCgl1575 gene in Corynebacterium glutamicum, or point mutation NCgl1575^{A1775T} and overexpression of NCgl1575 gene coding region are conducive to the increase of L-lysine production, while weakening or knocking out the gene is not conducive to the accumulation of L-lysine.

### Example 14: Construction of a Transformation Vector Pk18-PlysC^{(G(-45)A,G(-47)T)} Containing the Promoter Region of lysC Gene with Point Mutation

Based on the genome sequence of Corynebacterium glutamicum ATCC13032 published by NCBI, two pairs of primers for amplifying the sequences of lysC gene promoter region were designed and synthesized, and point mutation was introduced into the lysC gene promoter region (SEQ ID NO: 57) in the background of strain YP97158 by means of allelic replacement. The G at position −45 bp of lysC gene promoter region nucleotide sequence was changed to A, and the G at position −47 bp was changed to T (SEQ ID NO: 58).

Primers were designed as follows (synthesized by Shanghai Invitrogen Company):

P1″:
(SEQ ID NO: 59)
5′ CCGGAATTCG ACCAAGGATG AGGGCTTTG 3′;
(EcoR I)
P2″:
(SEQ ID NO: 60)
5′ AGTTACCCGC TCAATTATAC CTTTATAAAC 3′;
P3″:
(SEQ ID NO: 61)
5′ GTTTATAAAG GTATAATTGAGCGGGTAACT 3′;
and
P4″:
(SEQ ID NO: 62)
5′ ACATGCATGCGCGTACGCGAAGTGGCACAT 3′.
(Sph I)

Construction method: Corynebacterium glutamicum ATCC13032 was used as a template for PCR amplification with primers P1″ and P2″, P3″ and P4″, respectively.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL. The PCR amplification was carried out as follows: pre-denaturation for 5 min at 94° C., denaturation for 30 s at 94° C., annealing for 30 s at 52° C., extension for 40 s at 72° C. (30 cycles), and over extension for 10 min at 72° C.

Two DNA fragments with point mutation in size of 729 bp and 760 bp, respectively, were obtained (lysC promotor Up and lysC promotor Down fragments). After the above two DNA fragments were separated and purified by agarose gel electrophoresis, the purified two DNA fragments were used as templates, and P1″ and P4″ were used as primers to amplify a fragment with a length of about 1459 bp (Up Down fragment) by Overlap PCR.

PCR system: 10×Ex Taq Buffer 5 μL, dNTP Mixture (each 2.5 mM) 4 μL, Mg²⁺ (25 mM) 4 μL, primers (10 μM) each 2 μL, Ex Taq (5 U/μL) 0.25 μL, total volume 50 μL.

The PCR amplification was carried out as follows: pre-denaturation for 5 min at 94° C., denaturation for 30 s at 94° C., annealing for 30 s at 52° C., extension for 90 s at 72° C. (30 cycles), and over extension for 10 min at 72° C.

The above Up-Down fragment was separated and purified by agarose gel electrophoresis, and the fragment contained lysC gene promoter region and its upstream and downstream sequences, and both ends of the fragment contained EcoR I and Sph I enzyme digestion sites, respectively.

This DNA fragment causes the change of nucleotide guanine (G) at position −45 bp in the promoter region of YP97158 lysC gene to adenine (A), and the nucleotide guanine (G) at position −47 bp to thymine (T). The fragment was purified and recovered after double enzyme digestion (EcoR I/Sph I), and was linked with the shuttle plasmid pK18mobsacB (purchased from Addgene) after the same double enzyme digestion (EcoR I/Sph I) to obtain an allelic replacement plasmid pK18-PlysC^{(G(-45)A,G(-47)T)}, which contained a Kanamycin resistance marker. The vector pK18-PlysC^{(G(-45)A,G(-47)T)} was sent to the sequencing company for sequencing and identification, and the vector pK18-PlysC^{(G(-45)A,G(-47)T)} containing the correct point mutation was stored for use.

### Example 15: Construction of Engineering Strains Containing PlysC^{(G(-45)A,G(-47)T)} with Point Mutation

The allelic replacement plasmid pK18-PlysC^{(G(-45)A,G(-47)T)} was transformed into L-lysine production strain YP97158 by electric shock. The single colony produced by culturing was identified by primer P1″ and universal primer M13F, respectively, and the strains that can amplify a band in size of 1500 bp were positive strains. The positive strains were cultured on the medium containing 15% sucrose, and the single colony produced by culturing was cultured on the medium containing kanamycin and the medium without kanamycin, respectively; the strains that grew on the medium without kanamycin but did not grow on the medium containing kanamycin were further identified by PCR using the following primers (synthesized by Shanghai Invitrogen Company):

P5″:
(SEQ ID NO: 63)
5′ ATCAATATATGGTCTGTTTA 3′;
and
P6″:
(SEQ ID NO: 64)
5′ CTTGGTGGCAACGATCCGTT 3′

The above PCR amplification product was denatured at high temperature and subjected to ice bath followed by sscp electrophoresis (the plasmid pK18-PlysC^{(G(-45)A,G(-47)T)} amplification fragment was used as the positive control, YP97158 amplification fragment was used as the negative control, and the water was used as the blank control). Due to different fragment structures and electrophoresis positions, the strains whose electrophoresis positions are different from those of negative control fragments and are consistent with those of positive control fragments are the strains with successful allelic replacement. The target fragment of the positive strain was amplified by PCR again, and linked to the PMD19-T vector for sequencing. Through sequence alignment, the sequence in which base sequence is mutated verifies that the allelic replacement of the strain is successful, and it is named YPL-4-009.

### Example 16: L-Lysine Fermentation Experiment

The strain YPL-4-009 constructed in Example 15 and the original strain YP97158 were performed a fermentation experiment in the BLBIO-5GC-4-H fermentation tank (purchased from Shanghai Bailun Biotechnology Co., Ltd.) with the culture medium shown in Table 1 and the control process shown in Table 2. Each strain was repeated three times, and the results are shown in Table 8.

TABLE 8
L-Lysine fermentation experiment results

| | | L-Lysine production | Conversion |
|---|---|---|---|
| Strains | | (g/100 ml) | rate (%) |
| YP97158 | batch 1 | 18.7 | 64.1 |
| | batch 2 | 18.8 | 64.0 |
| | batch 3 | 18.8 | 63.7 |
| | mean | 18.8 | 63.9 |
| YPL-4-009 | batch 1 | 21.0 | 64.7 |
| | batch 2 | 20.9 | 64.6 |
| | batch 3 | 20.9 | 64.7 |
| | mean | 20.9 | 64.7 |

| | | |
|---|---|---|
| increased folds | 11.17% | 1.25% |

The above conversion rate = total mass of lysine/total consumption of glucose *100%

The results are shown in Table 8. The point mutation PlysC^{(G(-45)A,G(-47)T)} of lysC gene promoter in Corynebacterium glutamicum is contributive to the increase of the L-lysine production.

The embodiment of the invention has been described above. However, the present invention is not limited to the above embodiments. Any modification, equivalent replacement, improvement, etc. made within the spirit and principle of the invention shall be included in the protection scope of the invention.

What is claimed is:

1. A bacterium producing L-amino acid, wherein, the bacterium comprises overexpression of a polynucleotide encoding an amino acid sequence of SEQ ID NO: 3 or comprises expression or overexpression of a polynucleotide encoding the amino acid sequence of SEQ ID NO: 3 with a point mutation.

2. The bacterium as claimed in claim 1, wherein the point mutation of the polynucleotide encoding amino acid sequence of SEQ ID NO: 3 cause that arginine at position 334 of the amino acid sequence of SEQ ID NO: 3 is substituted by a terminator.

3. The bacterium as claimed in claim 1, wherein, the polynucleotide encoding the amino acid sequence of SEQ ID NO: 3 contains the nucleotide sequence of SEQ ID NO: 1.

4. The bacterium as claimed in claim 1, wherein, the polynucleotide sequence encoding the amino acid sequence of SEQ ID NO: 3 and having the point mutation is formed by a mutation of the 1000th base of the polynucleotide sequence shown in SEQ ID NO: 1.

5. The bacterium as claimed in claim 4, wherein the mutation includes the mutation of the 1000th base of the polynucleotide sequence shown in SEQ ID NO: 1 from cytosine (C) to thymine (T).

6. The bacterium as claimed in claim 4, wherein the polynucleotide sequence with the point mutation includes the polynucleotide sequence shown in SEQ ID NO: 2.

7. The bacterium as claimed in claim 1, wherein the improved expression is that the expression of the polynucleotide encoding the amino acid sequence of SEQ ID NO: 3 is enhanced or the polynucleotide encoding the amino acid sequence of SEQ ID NO: 3 has point mutations, or the polynucleotide encoding the amino acid sequence of SEQ ID NO: 3 has point mutations and the expression is enhanced.

8. The bacterium as claimed in claim 1, wherein, the microorganism comprises Corynebacterium glutamicum.

9. The bacterium as claimed in claim 5, wherein the Corynebacterium glutamicum is YP97158 or ATCC 13869.

10. A product selected from the group comprises:
(I) a polynucleotide sequence, wherein the polynucleotide sequence comprises a polynucleotide encoding an amino acid sequence shown in SEQ ID NO: 3 with arginine at position 334 thereof substituted by a terminator;
(II) an amino acid sequence comprises a sequence which is shown in SEQ ID NO:4;
(III) a recombinant vector, wherein, the recombinant vector comprises the polynucleotide sequence of said (I); and
(IV) a recombinant strain, wherein, the recombinant strain comprises the polynucleotide sequence of said (I).

11. The product as claimed in claim 10, wherein the polynucleotide sequence comprises a polynucleotide encoding an amino acid sequence shown in SEQ ID NO: 4.

12. The product as claimed in claim 10, wherein the polynucleotide sequence is formed by a mutation in the 1000th base of a polynucleotide sequence shown in SEQ ID NO: 1.

13. The product as claimed in claim 10, wherein the mutation comprises a mutation in the 485th base of the polynucleotide sequence as shown in SEQ ID NO: 1 from cytosine (C) to thymine (T).

14. The product as claimed in claim 10, wherein the polynucleotide sequence includes a polynucleotide sequence shown in SEQ ID NO: 2.

15. A method for producing L-amino acid, the method comprises culturing the bacterium of claim 1 and recovering L-amino acid from the culture.

16. A method for increasing production of L-glutamic acid in a microorganism, comprising increasing the expression of a polynucleotide encoding the amino acid sequence of SEQ ID NO: 3, or expressing or overexpressing a polynucleotide encoding the amino acid sequence of SEQ ID NO: 3 having a point mutation in the microorganism.

17. The method as claimed in claim 16, wherein the polynucleotide encoding the amino acid sequence of SEQ ID NO: 3 comprise the nucleotide sequence of SEQ ID NO: 1.

18. The method as claimed in claim 16, wherein the polynucleotide encoding the amino acid sequence of SEQ ID NO: 3 having a point mutation comprises a polynucleotide encoding an amino acid sequence set forth in SEQ ID NO: 4.

19. The method as claimed in claim 16, wherein the the microorganism is Corynebacterium glutamicum.


SEQUENCE LISTING (64 sequences)

SEQ ID NO: 1 - DNA, 1083 bp, Corynebacterium glutamicum
  source 1..1083: mol_type = other DNA
gtgtcacacaccgcgtccacaccgacgccagaggaatactccgcgcagcaacccagcacccagggcactcgcgttgagttccgcggcataaccaaagtctttagcaacaataaatctgctaaaaccaccgcgcttgataatgtcactctcaccgtagaacccggtgaggtaatcggcatcatcggttactctggcgccggcaagtccactcttgtccgcctcatcaatggccttgactcccccacgagcggttcgttgctgctcaacggcaccgacatcgtcggaatgcccgagtctaagctgcgtaaactgcgcagtaatatcggcatgattttccagcagttcaacctgttccagtcgcgtactgcggctggaaatgtggagtacccgctggaagttgccaagatggacaaggcagctcgtaaagctcgcgtgcaagaaatgctcgagttcgtcggcctgggcgacaaaggcaaaaactaccccgagcagctgtcgggcggccagaagcagcgcgtcggcattgcccgtgcactggccaccaatccaacgcttttgcttgccgacgaagccacctccgctttggacccagaaaccacccatgaagttctggagctgctgcgcaaggtaaaccgcgaactgggcatcaccatcgttgtgatcacccacgaaatggaagttgtgcgttccatcgcagacaaggttgctgtgatggaatccggcaaagttgtggaatacggcagcgtctacgaggtgttctccaatccacaaacacaggttgctcaaaagttcgtggccaccgcgctgcgtaacaccccagaccaagtggaatcggaagatctgcttagccatgagggacgtctgttcaccattgatctgactgaaacgtccggcttctttgcagcaaccgctcgtgctgccgaacaaggtgcttttgtcaacatcgttcacggtggcgtgaccaccttgcaacgccaatcatttggcaaaatgactgttcgactcaccggcaacaccgctgcgattgaagagttctatcaaaccttgaccaagaccacgaccatcaaggagatcacccgatga

SEQ ID NO: 2 - DNA, 1083 bp, synthetic construct
  misc_feature 1..1083: note = NCgl0609R334* ORF sequence
  source 1..1083: mol_type = other DNA
gtgtcacacaccgcgtccacaccgacgccagaggaatactccgcgcagcaacccagcacccagggcactcgcgttgagttccgcggcataaccaaagtctttagcaacaataaatctgctaaaaccaccgcgcttgataatgtcactctcaccgtagaacccggtgaggtaatcggcatcatcggttactctggcgccggcaagtccactcttgtccgcctcatcaatggccttgactcccccacgagcggttcgttgctgctcaacggcaccgacatcgtcggaatgcccgagtctaagctgcgtaaactgcgcagtaatatcggcatgattttccagcagttcaacctgttccagtcgcgtactgcggctggaaatgtggagtacccgctggaagttgccaagatggacaaggcagctcgtaaagctcgcgtgcaagaaatgctcgagttcgtcggcctgggcgacaaaggcaaaaactaccccgagcagctgtcgggcggccagaagcagcgcgtcggcattgcccgtgcactggccaccaatccaacgcttttgcttgccgacgaagccacctccgctttggacccagaaaccacccatgaagttctggagctgctgcgcaaggtaaaccgcgaactgggcatcaccatcgttgtgatcacccacgaaatggaagttgtgcgttccatcgcagacaaggttgctgtgatggaatccggcaaagttgtggaatacggcagcgtctacgaggtgttctccaatccacaaacacaggttgctcaaaagttcgtggccaccgcgctgcgtaacaccccagaccaagtggaatcggaagatctgcttagccatgagggacgtctgttcaccattgatctgactgaaacgtccggcttctttgcagcaaccgctcgtgctgccgaacaaggtgcttttgtcaacatcgttcacggtggcgtgaccaccttgcaacgccaatcatttggcaaaatgactgtttgactcaccggcaacaccgctgcgattgaagagttctatcaaaccttgaccaagaccacgaccatcaaggagatcacccgatga

SEQ ID NO: 3 - protein, 360 aa, Corynebacterium glutamicum
MSHTASTPTPEEYSAQQPSTQGTRVEFRGITKVFSNNKSAKTTALDNVTLTVEPGEVIGIIGYSGAGKSTLVRLINGLDSPTSGSLLLNGTDIVGMPESKLRKLRSNIGMIFQQFNLFQSRTAAGNVEYPLEVAKMDKAARKARVQEMLEFVGLGDKGKNYPEQLSGGQKQRVGIARALATNPTLLLADEATSALDPETTHEVLELLRKVNRELGITIVVITHEMEVVRSIADKVAVMESGKVVEYGSVYEVFSNPQTQVAQKFVATALRNTPDQVESEDLLSHEGRLFTIDLTETSGFFAATARAAEQGAFVNIVHGGVTTLQRQSFGKMTVRLTGNTAAIEEFYQTLTKTTTIKEITR

SEQ ID NO: 4 - protein, 333 aa, synthetic construct
  REGION 1..333: note = position 334 shown in SEQ ID NO: 3 is substituted by terminator
MSHTASTPTPEEYSAQQPSTQGTRVEFRGITKVFSNNKSAKTTALDNVTLTVEPGEVIGIIGYSGAGKSTLVRLINGLDSPTSGSLLLNGTDIVGMPESKLRKLRSNIGMIFQQFNLFQSRTAAGNVEYPLEVAKMDKAARKARVQEMLEFVGLGDKGKNYPEQLSGGQKQRVGIARALATNPTLLLADEATSALDPETTHEVLELLRKVNRELGITIVVITHEMEVVRSIADKVAVMESGKVVEYGSVYEVFSNPQTQVAQKFVATALRNTPDQVESEDLLSHEGRLFTIDLTETSGFFAATARAAEQGAFVNIVHGGVTTLQRQSFGKMTV

SEQ ID NO: 5 - DNA, 55 bp, synthetic construct
  misc_feature 1..55: note = Synthesized P1 primer
  source 1..55: mol_type = other DNA
cagtgccaagcttgcatgcctgcaggtcgactctagggacggcaacgtacataac

SEQ ID NO: 6 - DNA, 29 bp, synthetic construct
  misc_feature 1..29: note = Synthesized P2 primer
  source 1..29: mol_type = other DNA
gttgccggtgagtcaaacagtcattttgc

SEQ ID NO: 7 - DNA, 29 bp, synthetic construct
  misc_feature 1..29: note = Synthesized P3 primer
  source 1..29: mol_type = other DNA
gcaaaatgactgtttgactcaccggcaac

SEQ ID NO: 8 - DNA, 56 bp, synthetic construct
  misc_feature 1..56: note = Synthesized P4 primer
  source 1..56: mol_type = other DNA
cagctatgaccatgattacgaattcgagctcggtacccgcggctggaaatgtggag

SEQ ID NO: 9 - DNA, 18 bp, synthetic construct
  misc_feature 1..18: note = Synthesized P5 primer
  source 1..18: mol_type = other DNA
ctagccggttccagtcag

SEQ ID NO: 10 - DNA, 19 bp, synthetic construct
  misc_feature 1..19: note = Synthesized P6 primer
  source 1..19: mol_type = other DNA
ggacgtctgttcaccattg

SEQ ID NO: 11 - DNA, 55 bp, synthetic construct
  misc_feature 1..55: note = Synthesized P7 primer
  source 1..55: mol_type = other DNA
cagtgccaagcttgcatgcctgcaggtcgactctagaatgcgttctggactgagg

SEQ ID NO: 12 - DNA, 55 bp, synthetic construct; identical to SEQ ID NO: 11
  misc_feature 1..55: note = Synthesized P8 primer
  source 1..55: mol_type = other DNA

SEQ ID NO: 13 - DNA, 37 bp, synthetic construct
  misc_feature 1..37: note = Synthesized P9 primer
  source 1..37: mol_type = other DNA
catctgttctcggtgcaccagctgcgaggatcatctc

SEQ ID NO: 14 - DNA, 46 bp, synthetic construct
  misc_feature 1..46: note = Synthesized P10 primer
  source 1..46: mol_type = other DNA
gatttaattgcgccatctgattctggcaacaactccttccttgacc

SEQ ID NO: 15 - DNA, 49 bp, synthetic construct
  misc_feature 1..49: note = Synthesized P11 primer
  source 1..49: mol_type = other DNA
ggtcaaggaaggagttgttgccagaatcagatggcgcaattaaatcaag

SEQ ID NO: 16 - DNA, 60 bp, synthetic construct
  misc_feature 1..60: note = Synthesized P12 primer
  source 1..60: mol_type = other DNA
cagctatgaccatgattacgaattcgagctcggtacccgctatgacaccttcaacggatc

SEQ ID NO: 17 - DNA, 19 bp, synthetic construct
  misc_feature 1..19: note = Synthesized P13 primer
  source 1..19: mol_type = other DNA
tccaaggaagatacacgcc

SEQ ID NO: 18 - DNA, 18 bp, synthetic construct
  misc_feature 1..18: note = Synthesized P14 primer
  source 1..18: mol_type = other DNA
cgaaatggaagttgtgcg

SEQ ID NO: 19 - DNA, 19 bp, synthetic construct
  misc_feature 1..19: note = Synthesized P15 primer
  source 1..19: mol_type = other DNA
cgatgatgccgattacctc

SEQ ID NO: 20 - DNA, 18 bp, synthetic construct
  misc_feature 1..18: note = Synthesized P16 primer
  source 1..18: mol_type = other DNA
cgttggaatcttgcgttg

SEQ ID NO: 21 - DNA, 55 bp, synthetic construct
  misc_feature 1..55: note = Synthesized P17 primer
  source 1..55: mol_type = other DNA
gcttgcatgcctgcaggtcgactctagaggatcccccagctgcgaggatcatctc

SEQ ID NO: 22 - DNA, 54 bp, synthetic construct
  misc_feature 1..54: note = Synthesized P18 primer
  source 1..54: mol_type = other DNA
atcaggctgaaaatcttctctcatccgccaaaaccaacaactccttccttgacc

SEQ ID NO: 23 - DNA, 54 bp, synthetic construct
  misc_feature 1..54: note = Synthesized P19 primer
  source 1..54: mol_type = other DNA
cagtgccaagcttgcatgcctgcaggtcgactctagaatgaatgggatgggtcg

SEQ ID NO: 24 - DNA, 37 bp, synthetic construct
  misc_feature 1..37: note = Synthesized P20 primer
  source 1..37: mol_type = other DNA
catcatcggttactctggccgaaatggaagttgtgcg

SEQ ID NO: 25 - DNA, 37 bp, synthetic construct
  misc_feature 1..37: note = Synthesized P21 primer
  source 1..37: mol_type = other DNA
cgcacaacttccatttcggccagagtaaccgatgatg

SEQ ID NO: 26 - DNA, 58 bp, synthetic construct
  misc_feature 1..58: note = Synthesized P22 primer
  source 1..58: mol_type = other DNA
cagctatgaccatgattacgaattcgagctcggtaccccaacaactccttccttgacc

SEQ ID NO: 27 - DNA, 18 bp, synthetic construct
  misc_feature 1..18: note = Synthesized P23 primer
  source 1..18: mol_type = other DNA
aatgaatgggatgggtcg

SEQ ID NO: 28 - DNA, 20 bp, synthetic construct
  misc_feature 1..20: note = Synthesized P24 primer
  source 1..20: mol_type = other DNA
caacaactccttccttgacc

SEQ ID NO: 29 - DNA, 2679 bp, Corynebacterium glutamicum
  source 1..2679: mol_type = other DNA
atggcagaatcaaacgctatggaccgggcacaaatctctgcactgctagatagagcacagcacacaatcaaccttgccgaacaagcaaacaacgtgctccgactgttgaaaacacccggaacggccacagtaggggacaacgggacactcggcaccgatacctatctgatcccatcccgcaacatcacctggcctgacaacctgtatgtcaacgtctttctagacggcatgaatgcagaagccacccttaccgattacgtcgcatcagtcgcttcgatcccacgcctatgccagatcatcaacgagggccaaggcggcatgttccgcagactattcaaccccaccaaggtccaagccggcgaccaagctgtcttcgacctcatggtcaaactcgacgagatttcatctaccacccacgaagtctcccgcatgctcgagggcgtccacgctgcccgcacccgccaacaacaaggcgttgcacttttcccaggtattcatggagtgggagagcgctacatcgaacgcgcacaacaggtactcgcctcagccctcggtatcgctggattcggtgccgaaccctgggacggacatacccttgcccaagcgcgccgggtagtccaacgctacgcccaagatcctaactccgaataccggctgaaaagcgaagccgagaaacacctcacatccatcaacgagctccgcgtacagatactcctcgaacaactccccgttgatgccctacgcatggctaccgaccaccgcctgcgctttggatccctcgattccatccacgtcgcaaccgtcgccgacgtcctaaaaacacacacctccatcctcaccaccgtgcaaggtatcggcgcccaaaccgcggggcggatgaaagccgcagcagaaacactcaaacaagaagcactacgccgccaaaacacctccatcggcgacgaacctacccaacccgccatgcgtctaatcaacgtgctggcccgcttcgaccaaaccgaaaccatcacgcccgaagaacgcgcccgccgcacccgcgtcatcgactacgtagaacacatacccccaagcctcgacccctacatcgtcatcaacccagcaacgcctgagttcaacaacttcaccgacgacctccgctggatcgacgcaaaccccaacctcttccacccacaaacaatcaccaccccacccgccgacatctgggacgactacatctcccgtcccgctcactaccaaggcctgctagccacgctgctcggccgcgacatcgaaggcgcagacgaactcctcgacgccaccaccctccaaaaaatcagagacctcaccctcgacaaaactcatctcaccgacctccacctccgcggataccaatcattcggcgcccgcttcgccatcatccaaaagaaaaccctcctcggcgacgacatgggactcggcaaaacagtccaagccctctccgcagctgcacaccttgccgccaccgaaaaagacttccgcaccctcgtcgtcgtacccgcatccgtcattgttaactggacccgcgaatgcaaacgcttcctcaacctccccgtattcatcgcccacggagacaacaaacaagacgccatcaacgcctggtctaacaccaacggaatcgcaatctgcacctacgacggcgtccgcaccatggacatccccgcgccgggtctggtcattgccgatgaagcccacctgatcaaaaacccctccaccaaacgcacccaagcactgcgcaaacttatcgacgccgccccatacacccttctgatgaccggcacaccactagaaaacaaagtggaagagtttgtaaatctcgtgcgctacatccaaccggagctgatcacccgtggcatgtccaaaatgcaggccgagaatttccgcgagcgcatcgcaccagcctatctgcgcagaaatcaagctgatgtgcttgacgaactcccagagcgcaccgactccatcgactggatcgacctcaccccagaagaccgcagcgcctacgacgaccaagtccgccaaggcagctggatgggcatgcgccgctccgccatgctctcaccaacaccacgcctaacttccgcaaaaatgcaacgcatcctagaactcttcgaagaagcagaagaacacggccgcaaagccctcatcttcacctacttcctcgacgtcctcgacgaactggaaaagcatctaggcgagcgcgtcatcggccgcatttccggcgacgtgccagccaccaagcgccaattgcttgtcgacgccctgtcccactccaaacccggatccgccctcattgcccaaatcaccgccgggggagtaggcctaaacatccaatccgcgagcctatgcattatttgtgaacctcaagtaaagccaaccatcgaacagcaggccgtcgcccgagtccaccgcatgggccaaaccgccaccgtccaagtccaccgactcatcggcgacgaaaccgcagacgaacgcatgctagaaatcctggcaggcaaaactcacgtcttcgacgtctacgcccggctatctgaaaccgcagagattccagatgctgtggatatcactgaatcacagctggcagcacgggttattgatgaggagcgtgcacggttagggcttactgaatccactggccctaaagatgaagaaacggccttaagctag

SEQ ID NO: 30 - DNA, 2679 bp, synthetic construct
  misc_feature 1..2679: note = NCgl1575A1775T ORF sequence
  source 1..2679: mol_type = other DNA
atggcagaatcaaacgctatggaccgggcacaaatctctgcactgctagatagagcacagcacacaatcaaccttgccgaacaagcaaacaacgtgctccgactgttgaaaacacccggaacggccacagtaggggacaacgggacactcggcaccgatacctatctgatcccatcccgcaacatcacctggcctgacaacctgtatgtcaacgtctttctagacggcatgaatgcagaagccacccttaccgattacgtcgcatcagtcgcttcgatcccacgcctatgccagatcatcaacgagggccaaggcggcatgttccgcagactattcaaccccaccaaggtccaagccggcgaccaagctgtcttcgacctcatggtcaaactcgacgagatttcatctaccacccacgaagtctcccgcatgctcgagggcgtccacgctgcccgcacccgccaacaacaaggcgttgcacttttcccaggtattcatggagtgggagagcgctacatcgaacgcgcacaacaggtactcgcctcagccctcggtatcgctggattcggtgccgaaccctgggacggacatacccttgcccaagcgcgccgggtagtccaacgctacgcccaagatcctaactccgaataccggctgaaaagcgaagccgagaaacacctcacatccatcaacgagctccgcgtacagatactcctcgaacaactccccgttgatgccctacgcatggctaccgaccaccgcctgcgctttggatccctcgattccatccacgtcgcaaccgtcgccgacgtcctaaaaacacacacctccatcctcaccaccgtgcaaggtatcggcgcccaaaccgcggggcggatgaaagccgcagcagaaacactcaaacaagaagcactacgccgccaaaacacctccatcggcgacgaacctacccaacccgccatgcgtctaatcaacgtgctggcccgcttcgaccaaaccgaaaccatcacgcccgaagaacgcgcccgccgcacccgcgtcatcgactacgtagaacacatacccccaagcctcgacccctacatcgtcatcaacccagcaacgcctgagttcaacaacttcaccgacgacctccgctggatcgacgcaaaccccaacctcttccacccacaaacaatcaccaccccacccgccgacatctgggacgactacatctcccgtcccgctcactaccaaggcctgctagccacgctgctcggccgcgacatcgaaggcgcagacgaactcctcgacgccaccaccctccaaaaaatcagagacctcaccctcgacaaaactcatctcaccgacctccacctccgcggataccaatcattcggcgcccgcttcgccatcatccaaaagaaaaccctcctcggcgacgacatgggactcggcaaaacagtccaagccctctccgcagctgcacaccttgccgccaccgaaaaagacttccgcaccctcgtcgtcgtacccgcatccgtcattgttaactggacccgcgaatgcaaacgcttcctcaacctccccgtattcatcgcccacggagacaacaaacaagacgccatcaacgcctggtctaacaccaacggaatcgcaatctgcacctacgacggcgtccgcaccatggacatccccgcgccgggtctggtcattgccgatgaagcccacctgatcaaaaacccctccaccaaacgcacccaagcactgcgcaaacttatcgacgccgccccattcacccttctgatgaccggcacaccactagaaaacaaagtggaagagtttgtaaatctcgtgcgctacatccaaccggagctgatcacccgtggcatgtccaaaatgcaggccgagaatttccgcgagcgcatcgcaccagcctatctgcgcagaaatcaagctgatgtgcttgacgaactcccagagcgcaccgactccatcgactggatcgacctcaccccagaagaccgcagcgcctacgacgaccaagtccgccaaggcagctggatgggcatgcgccgctccgccatgctctcaccaacaccacgcctaacttccgcaaaaatgcaacgcatcctagaactcttcgaagaagcagaagaacacggccgcaaagccctcatcttcacctacttcctcgacgtcctcgacgaactggaaaagcatctaggcgagcgcgtcatcggccgcatttccggcgacgtgccagccaccaagcgccaattgcttgtcgacgccctgtcccactccaaacccggatccgccctcattgcccaaatcaccgccgggggagtaggcctaaacatccaatccgcgagcctatgcattatttgtgaacctcaagtaaagccaaccatcgaacagcaggccgtcgcccgagtccaccgcatgggccaaaccgccaccgtccaagtccaccgactcatcggcgacgaaaccgcagacgaacgcatgctagaaatcctggcaggcaaaactcacgtcttcgacgtctacgcccggctatctgaaaccgcagagattccagatgctgtggatatcactgaatcacagctggcagcacgggttattgatgaggagcgtgcacggttagggcttactgaatccactggccctaaagatgaagaaacggccttaagctag

SEQ ID NO: 31 - protein, 892 aa, Corynebacterium glutamicum
MAESNAMDRAQISALLDRAQHTINLAEQANNVLRLLKTPGTATVGDNGTLGTDTYLIPSRNITWPDNLYVNVFLDGMNAEATLTDYVASVASIPRLCQIINEGQGGMFRRLFNPTKVQAGDQAVFDLMVKLDEISSTTHEVSRMLEGVHAARTRQQQGVALFPGIHGVGERYIERAQQVLASALGIAGFGAEPWDGHTLAQARRVVQRYAQDPNSEYRLKSEAEKHLTSINELRVQILLEQLPVDALRMATDHRLRFGSLDSIHVATVADVLKTHTSILTTVQGIGAQTAGRMKAAAETLKQEALRRQNTSIGDEPTQPAMRLINVLARFDQTETITPEERARRTRVIDYVEHIPPSLDPYIVINPATPEFNNFTDDLRWIDANPNLFHPQTITTPPADIWDDYISRPAHYQGLLATLLGRDIEGADELLDATTLQKIRDLTLDKTHLTDLHLRGYQSFGARFAIIQKKTLLGDDMGLGKTVQALSAAAHLAATEKDFRTLVVVPASVIVNWTRECKRFLNLPVFIAHGDNKQDAINAWSNTNGIAICTYDGVRTMDIPAPGLVIADEAHLIKNPSTKRTQALRKLIDAAPYTLLMTGTPLENKVEEFVNLVRYIQPELITRGMSKMQAENFRERIAPAYLRRNQADVLDELPERTDSIDWIDLTPEDRSAYDDQVRQGSWMGMRRSAMLSPTPRLTSAKMQRILELFEEAEEHGRKALIFTYFLDVLDELEKHLGERVIGRISGDVPATKRQLLVDALSHSKPGSALIAQITAGGVGLNIQSASLCIICEPQVKPTIEQQAVARVHRMGQTATVQVHRLIGDETADERMLEILAGKTHVFDVYARLSETAEIPDAVDITESQLAARVIDEERARLGLTESTGPKDEETALS

SEQ ID NO: 32 - protein, 892 aa, synthetic construct
  REGION 1..892: note = NCgl1575 Y592F coding protein sequence
MAESNAMDRAQISALLDRAQHTINLAEQANNVLRLLKTPGTATVGDNGTLGTDTYLIPSRNITWPDNLYVNVFLDGMNAEATLTDYVASVASIPRLCQIINEGQGGMFRRLFNPTKVQAGDQAVFDLMVKLDEISSTTHEVSRMLEGVHAARTRQQQGVALFPGIHGVGERYIERAQQVLASALGIAGFGAEPWDGHTLAQARRVVQRYAQDPNSEYRLKSEAEKHLTSINELRVQILLEQLPVDALRMATDHRLRFGSLDSIHVATVADVLKTHTSILTTVQGIGAQTAGRMKAAAETLKQEALRRQNTSIGDEPTQPAMRLINVLARFDQTETITPEERARRTRVIDYVEHIPPSLDPYIVINPATPEFNNFTDDLRWIDANPNLFHPQTITTPPADIWDDYISRPAHYQGLLATLLGRDIEGADELLDATTLQKIRDLTLDKTHLTDLHLRGYQSFGARFAIIQKKTLLGDDMGLGKTVQALSAAAHLAATEKDFRTLVVVPASVIVNWTRECKRFLNLPVFIAHGDNKQDAINAWSNTNGIAICTYDGVRTMDIPAPGLVIADEAHLIKNPSTKRTQALRKLIDAAPFTLLMTGTPLENKVEEFVNLVRYIQPELITRGMSKMQAENFRERIAPAYLRRNQADVLDELPERTDSIDWIDLTPEDRSAYDDQVRQGSWMGMRRSAMLSPTPRLTSAKMQRILELFEEAEEHGRKALIFTYFLDVLDELEKHLGERVIGRISGDVPATKRQLLVDALSHSKPGSALIAQITAGGVGLNIQSASLCIICEPQVKPTIEQQAVARVHRMGQTATVQVHRLIGDETADERMLEILAGKTHVFDVYARLSETAEIPDAVDITESQLAARVIDEERARLGLTESTGPKDEETALS

SEQ ID NO: 33 - DNA, 56 bp, synthetic construct
  misc_feature 1..56: note = Synthesized P1' primer
  source 1..56: mol_type = other DNA
cagtgccaagcttgcatgcctgcaggtcgactctagtgcgttcgtctgcggtttcg

SEQ ID NO: 34 - DNA, 30 bp, synthetic construct
  misc_feature 1..30: note = Synthesized P2' primer
  source 1..30: mol_type = other DNA
atcgacgccgccccattcacccttctgatg

SEQ ID NO: 35 - DNA, 30 bp, synthetic construct
  misc_feature 1..30: note = Synthesized P3' primer
  source 1..30: mol_type = other DNA
catcagaagggtgaatggggcggcgtcgat

SEQ ID NO: 36 - DNA, 57 bp, synthetic construct
  misc_feature 1..57: note = Synthesized P4' primer
  source 1..57: mol_type = other DNA
cagctatgaccatgattacgaattcgagctcggtacccaagcctcgacccctacatc

SEQ ID NO: 37 - DNA, 20 bp, synthetic construct
  misc_feature 1..20: note = Synthesized P5' primer
  source 1..20: mol_type = other DNA
cacatcagcttgatttctgc

SEQ ID NO: 38 - DNA, 20 bp, synthetic construct
  misc_feature 1..20: note = Synthesized P6' primer
  source 1..20: mol_type = other DNA
ggtcattgccgatgaagccc

SEQ ID NO: 39 - DNA, 55 bp, synthetic construct; identical to SEQ ID NO: 11
  misc_feature 1..55: note = Synthesized P7' primer
  source 1..55: mol_type = other DNA

SEQ ID NO: 40 - DNA, 36 bp, synthetic construct
  misc_feature 1..36: note = Synthesized P8' primer
  source 1..36: mol_type = other DNA
gaaacggccttaagctaggtgcaccgagaacagatg

SEQ ID NO: 41 - DNA, 36 bp, synthetic construct
  misc_feature 1..36: note = Synthesized P9' primer
  source 1..36: mol_type = other DNA
catctgttctcggtgcacctagcttaaggccgtttc

SEQ ID NO: 42 - DNA, 39 bp, synthetic construct
  misc_feature 1..39: note = Synthesized P10' primer
  source 1..39: mol_type = other DNA
cttgatttaattgcgccatcaagcttttcccgcccggtt

SEQ ID NO: 43 - DNA, 39 bp, synthetic construct
  misc_feature 1..39: note = Synthesized P11' primer
  source 1..39: mol_type = other DNA
aaccgggcgggaaaagcttgatggcgcaattaaatcaag

SEQ ID NO: 44 - DNA, 60 bp, synthetic construct; identical to SEQ ID NO: 16
  misc_feature 1..60: note = Synthesized P12' primer
  source 1..60: mol_type = other DNA

SEQ ID NO: 45 - DNA, 19 bp, synthetic construct
  misc_feature 1..19: note = Synthesized P13' primer
  source 1..19: mol_type = other DNA
tccaaggaagatacacgcc

SEQ ID NO: 46 - DNA, 20 bp, synthetic construct
  misc_feature 1..20: note = Synthesized P14' primer
  source 1..20: mol_type = other DNA
cttctgatgaccggcacacc

SEQ ID NO: 47 - DNA, 20 bp, synthetic construct
  misc_feature 1..20: note = Synthesized P15' primer
  source 1..20: mol_type = other DNA
tagtcgatgacgcgggtgcg

SEQ ID NO: 48 - DNA, 18 bp, synthetic construct
  misc_feature 1..18: note = Synthesized P16' primer
  source 1..18: mol_type = other DNA
cgttggaatcttgcgttg

SEQ ID NO: 49 - DNA, 54 bp, synthetic construct
  misc_feature 1..54: note = Synthesized P17' primer
  source 1..54: mol_type = other DNA
gcttgcatgcctgcaggtcgactctagaggatccccctagcttaaggccgtttc

SEQ ID NO: 50 - DNA, 53 bp, synthetic construct
  misc_feature 1..53: note = Synthesized P18' primer
  source 1..53: mol_type = other DNA
atcaggctgaaaatcttctctcatccgccaaaacaagcttttcccgcccggtt

SEQ ID NO: 51 - DNA, 56 bp, synthetic construct
  misc_feature 1..56: note = Synthesized P19' primer
  source 1..56: mol_type = other DNA
cagtgccaagcttgcatgcctgcaggtcgactctagaccggcgcagatgccaacgc

SEQ ID NO: 52 - DNA, 37 bp, synthetic construct
  misc_feature 1..37: note = Synthesized P20' primer
  source 1..37: mol_type = other DNA
cccagaactgaaggtctaattgcctaaggccggaatt

SEQ ID NO: 53 - DNA, 37 bp, synthetic construct
  misc_feature 1..37: note = Synthesized P21' primer
  source 1..37: mol_type = other DNA
aattccggccttaggcaattagaccttcagttctggg

SEQ ID NO: 54 - DNA, 56 bp, synthetic construct
  misc_feature 1..56: note = Synthesized P22' primer
  source 1..56: mol_type = other DNA
cagctatgaccatgattacgaattcgagctcggtacccgcttgatgaaggctccag

SEQ ID NO: 55 - DNA, 20 bp, synthetic construct
  misc_feature 1..20: note = Synthesized P23' primer
  source 1..20: mol_type = other DNA
accggcgcagatgccaacgc

SEQ ID NO: 56 - DNA, 18 bp, synthetic construct
  misc_feature 1..18: note = Synthesized P24' primer
  source 1..18: mol_type = other DNA
gcttgatgaaggctccag

SEQ ID NO: 57 - DNA, 242 bp, Corynebacterium glutamicum
  source 1..242: mol_type = other DNA
ttcagggtagttgactaaagagttgctcgcgaagtagcacctgtcacttttgtctcaaatattaaatcgaatatcaatatatggtctgtttattggaacgcgtcccagtggctgagacgcatccgctaaagccccaggaaccctgtgcagaaagaaaacactcctctggctaggtagacacagtttataaaggtagagttgagcgggtaactgtcagcacgtagatcgaaaggtgcacaaag

SEQ ID NO: 58 - DNA, 242 bp, synthetic construct
  misc_feature 1..242: note = mutated promotor sequence
  source 1..242: mol_type = other DNA
ttcagggtagttgactaaagagttgctcgcgaagtagcacctgtcacttttgtctcaaatattaaatcgaatatcaatatatggtctgtttattggaacgcgtcccagtggctgagacgcatccgctaaagccccaggaaccctgtgcagaaagaaaacactcctctggctaggtagacacagtttataaaggtataattgagcgggtaactgtcagcacgtagatcgaaaggtgcacaaag

SEQ ID NO: 59 - DNA, 29 bp, synthetic construct
  misc_feature 1..29: note = Synthesized P1'' primer
  source 1..29: mol_type = other DNA
ccggaattcgaccaaggatgagggctttg

SEQ ID NO: 60 - DNA, 30 bp, synthetic construct
  misc_feature 1..30: note = Synthesized P2'' primer
  source 1..30: mol_type = other DNA
agttacccgctcaattatacctttataaac

SEQ ID NO: 61 - DNA, 30 bp, synthetic construct
  misc_feature 1..30: note = Synthesized P3'' primer
  source 1..30: mol_type = other DNA
gtttataaaggtataattgagcgggtaact

SEQ ID NO: 62 - DNA, 30 bp, synthetic construct
  misc_feature 1..30: note = Synthesized P4'' primer
  source 1..30: mol_type = other DNA
acatgcatgcgcgtacgcgaagtggcacat

SEQ ID NO: 63 - DNA, 20 bp, synthetic construct
  misc_feature 1..20: note = Synthesized P5'' primer
  source 1..20: mol_type = other DNA
atcaatatatggtctgttta

SEQ ID NO: 64 - DNA, 20 bp, synthetic construct
  misc_feature 1..20: note = Synthesized P6'' primer
  source 1..20: mol_type = other DNA
cttggtggcaacgatccgtt